(19) United States
(12) Patent Application Publication
(10) Pub. No.: US 2026/0224847 A1
(43) Pub. Date: Aug. 6, 2026

(54) CATHETERS AND SYSTEMS FOR DELIVERY OF GAS-ENRICHED LIQUID INTO A PATIENT

(71) Applicants: Zoll Circulation, Inc., San Jose, CA (US); ZOLL Medical Corporation, Chelmsford, MA (US)

(72) Inventors: Stephen Elwood Myrick, Tustin, CA (US); Kelsey Winward, San Clemente, CA (US)

(21) Appl. No.: 19/153,264

(22) PCT Filed: Feb. 2, 2024
(86) PCT No.: PCT/US2024/014124

§ 371 Date: Aug. 1, 2025

Related U.S. Application Data
(60) Provisional application No. 63/482,961, filed on Feb. 2, 2023.

(51) Int. Cl. A61M 25/00; A61M 25/09

(52) U.S. Cl. CPC A61M 25/0068; A61M 25/09; A61M 2025/0073; A61M 2202/0208; A61M 2230/205

(57) ABSTRACT

A catheter is configured to be inserted into a vasculature of a patient for delivery of a gas-enriched liquid. The catheter includes a catheter body and one or more capillaries coupled to the catheter body and configured to receive a gas-enriched liquid from a gas-enriched liquid source. An end surface of the catheter includes a first end of the catheter body and a first end of the one or more capillaries. The first end of the one or more capillaries has an aperture through which the gas-enriched liquid can flow. The first end of the one or more capillaries being flush with the first end of the catheter body wherein the end surface of the catheter is smooth and includes the aperture of the one or more capillaries.

### CLAIM OF PRIORITY

This application claims priority under 35 U.S.C. § 119(e) to U.S. Patent Application Ser. No. 63/482,961, filed on Feb. 2, 2023, the entire contents of which are hereby incorporated by reference.

### TECHNICAL FIELD

The disclosure relates to catheters and systems for delivery of gas-enriched liquid into a patient.

### BACKGROUND

Gas-enriched liquids are desirable in a wide variety of applications. However, at ambient pressure, the relatively low solubility of many gases, such as oxygen or nitrogen, within a liquid, such as water, produces a relatively low concentration of the dissolved gas in the liquid. One method of obtaining an increase in the gas concentration level without significant increase in liquid volume involves an injection and mixing of a gas-enriched liquid, such as a gas-supersaturated liquid, into a liquid of interest. A liquid can be gas-enriched at high pressure.

Conventional methods for the delivery of oxygenated blood or oxygen-enriched liquids to tissues and bodily liquids involve the use of extracorporeal circuits for blood oxygenation. Extracorporeal circuits require withdrawing blood from a patient, circulating the blood through an oxygenator to increase blood oxygen concentration, and then delivering the blood back to the patient.

### SUMMARY

A catheter and a system including the catheter are configured to deliver gas-enriched liquids to a vasculature of a patient. The catheter includes at least one capillary that delivers a stream of the gas-enriched liquid into a vasculature of a patient. The capillary is configured to deliver the gas-enriched liquid to mix with the blood of the patient. The stream of the gas-enriched liquid, which can include super-saturated oxygen (SSO₂) may be delivered while preventing or minimizing nucleation or bubble formation (e.g., without clinically significant gas emboli) inside the vasculature. In certain implementations, oxygen enriched liquid or solution, e.g., supersaturated oxygen liquid or solution, may include liquid having a dissolved O₂ concentration of 0.1 ml O₂/ml liquid (STP) or greater or 0.1-6 ml O₂/ml liquid (STP) or 0.2-3 ml O₂/ml liquid (STP) (e.g., without clinically significant gas emboli). For example, 1.0 milliliter (ml) or more of gas or outgassing of the dissolved oxygen, from the gas-enriched liquid in a patient's vasculature or tissue may be considered a clinically significant amount of gas emboli or outgassing. In certain implementations, the catheter may deliver a stream of gas-enriched (e.g., oxygen-enriched) liquid with a maximum of 0.99 ml of gas or outgassing of the dissolved oxygen from the gas-enriched liquid, e.g., about 0.001 to 0.99 ml of gas or outgassing of the dissolved oxygen from the gas-enriched liquid, in the stream or forming inside the vasculature or tissue. In certain implementations, the catheter may deliver a stream of gas-enriched (e.g., oxygen-enriched) liquid with less than 0.01 ml of gas or outgassing of the dissolved oxygen from the gas-enriched liquid, e.g., about 0.001 to 0.009 ml of gas or outgassing of the dissolved oxygen from the gas-enriched liquid, in the stream or forming inside the vasculature or tissue, or no more than 0.01 ml of gas or outgassing of the dissolved oxygen from the gas-enriched liquid. In certain implementations, the system or catheter may include a bubble detector which may initiate a shutdown of the infusion of oxygen enriched liquid if an accumulated volume of 10 microliters of gas or outgassing of the dissolved oxygen from the gas-enriched liquid (or 0.01 ml) is detected. For example, if 0.01 ml of gas or outgassing of the dissolved oxygen from the gas-enriched liquid is detected during an infusion lasting up to 120 mins in duration or about 60-120 min, shutdown may be initiated.

In certain embodiments, the catheters described herein, are configured to deliver oxygen dissolved in a solution. The catheters described herein can deliver the oxygen dissolved in the solution at about 500-1000 pounds per square inch (PSI). In an example, a catheter may deliver about 0.2-3 ml O₂/ml liquid at 100-1500 PSI to prevent or minimize outgassing. In another example, the gas-enriched liquid is delivered at 500-1000 PSI with an oxygen concentration between 1 ml O₂/1ml solution to 2 ml O₂/1ml solution. In some embodiments, the catheter minimizes outgassing, as previously described. While outgassing can be limited to 0 ml, a maximum amount of outgassing can be less than 1 or 0.99 ml of oxygen outgassing from the solution into the bloodstream or tissue. In another embodiment, the catheter limits outgassing to lesson more than 0.01 ml of oxygen outgassing from the solution.

The stream of the gas-enriched liquid may also be delivered such that mixing of the gas-enriched liquid and blood occurs at sufficient distance from the catheter end surface to prevent or minimize recirculation from occurring near and around the end of the catheter. Minimizing or reducing recirculation reduces or eliminates dead zones that include localized vortices of blood. These localized vortices of blood may facilitate blood clotting on the catheter surface. The stream of gas-enriched liquid delivers the oxygen into the bloodstream of the patient and causes the oxygen to perfuse into the patient's tissue. The catheter can include a plurality of capillaries (e.g., two or more capillaries). The capillaries may be angled near an end of the catheter so that the streams of gas-enriched liquid delivered by the capillaries intersect and mix within the vasculature of the patient without nucleation or recirculation or a minimized nucleation (e.g., without clinically significant gas emboli) or recirculation inside the vasculature.

The catheter includes an end that may expose the capillaries to the vasculature of the patient. The capillaries may be flush with the end of the catheter to form a solid, continuous surface except for apertures for the capillaries. A body of the catheter that supports the capillaries and ends of the capillaries may be polished to form the end of the catheter. The capillaries may not extend from the end of the catheter body.

The systems, devices, and processes described herein may provide one or more of the following advantages. A catheter having a smooth end can be positioned anywhere in a vasculature of a patient. The catheter may be polished to have a smooth, continuous end surface that is exposed to the bloodstream of the patient in the vasculature of the patient. In implementations in which no capillaries extend from the end of the catheter, the catheter can be positioned anywhere in the vasculature with a low risk or no risk of damaging the vasculature of the patient. The smooth surface of the catheter may reduce or eliminate formation of blood clots on the surface of the catheter. The smooth surface of the catheter may reduce or eliminate recirculation of blood near and around the end of the catheter. The smooth surface of the catheter may reduce or eliminate dead zones that include localized vortices of blood.

One or more of these advantages are enabled by the aspects and embodiments of the systems, devices, and processes described as follows.

In some implementations, a catheter is configured to be inserted into a vasculature of a patient for delivery of a gas-enriched liquid. The catheter includes a catheter body. The catheter includes two or more capillaries coupled to the catheter body and configured to receive a gas-enriched liquid from a gas-enriched liquid source. An end surface of the catheter includes a first end of the catheter body and first ends of each of the two or more capillaries. The first ends of the two or more capillaries include apertures through which the gas-enriched liquid can flow. The first ends of the two or more capillaries are flush with the first end of the catheter body. The end surface of the catheter is smooth and includes the apertures of the two or more capillaries. The two or more capillaries are in fluid communication with the vasculature of the patient when the catheter body is inserted into the vasculature of the patient. The two or more capillaries are configured to dispense respective streams of the gas-enriched liquid directly into the vasculature of the patient. The first ends of the two or more capillaries are positioned relative to one another to cause the streams of the gas-enriched liquid to intersect and mix with blood of the patient in a region beyond an end (the first end) of the catheter body.

The capillaries may be flush with the end of the catheter to form a solid, continuous surface except for apertures for the capillaries. A body of the catheter that supports the capillaries and ends of the capillaries may be polished to form the end of the catheter. The capillaries may not extend from the end of the catheter body. The capillaries may not extend beyond the end of the catheter body. The capillaries may not extend beyond a flat surface which defines the end of the catheter body. The ends of the capillaries may terminate at the end of the catheter body. The apertures of the capillaries may terminate at the end of the catheter body. The opening of the apertures of the capillaries may be flush with a first end of the catheter body. The capillaries at the first end of the catheter may be provided entirely within the catheter body.

In some implementations, the end surface is a flat surface. In some implementations, the end surface is a concave surface. In some implementations, the first ends of the two or more capillaries are beveled. In some implementations, the catheter body includes slots for positioning the two or more capillaries, the slots positioning the two or more capillaries with respect to one another for causing the streams of the gas-enriched liquid to intersect.

In some implementations, the two or more capillaries are coupled to the catheter body by an adhesive. In some implementations, a capillary of the two or more capillaries comprises a glass wall that is coated in a polyamide. In some implementations, the glass wall is polished to form a first end that is a portion of the end surface. In some implementations, the gas-enriched liquid comprises a supersaturated oxygen enriched liquid.

In some implementations, the catheter includes a lumen configured to receive a guide wire for positioning the catheter with respect to the vasculature of the patient. In some implementations, the catheter includes a pressure sensor disposed within the lumen, wherein the pressure sensor is configured to obtain one or more pressure measurements.

In some implementations, a largest diameter of the catheter, e.g., the end surface, is between 1 to 14 French. In some implementations, a largest diameter of the catheter is between 4-7 French. In some implementations, a largest diameter of the catheter is between 1 to 3 French. In some implementations, a largest diameter of the catheter is between 1 to 7 French. In some implementations, a diameter of the end surface is between 1 and 14 French. In some embodiments, a diameter of the end surface is 6.5 French. In some implementations, a largest diameter of the catheter, e.g., the end surface, is between 1 and 14 French.

In some implementations, the catheter includes a lumen extending through the catheter body. The lumen is configured to be in fluid communication with the vasculature of the patient when the catheter is inserted into the vasculature of the patient. The lumen is configured to perform at least one of receiving an additional sample of the blood from the vasculature of the patient and or measuring an additional parameter of the blood.

In some implementations, the catheter body is configured to position the two or more capillaries such that the streams of the gas-enriched liquid intersect a longitudinal axis extending through a center of the catheter.

In some implementations, the catheter body is configured to position the two or more capillaries such that the streams of the gas-enriched liquid do not intersect a longitudinal axis extending through a center of the catheter. In some implementations, the two or more capillaries form respective lumens that are angled with respect to a longitudinal axis of the catheter body at an angle of about 10-30 degrees, for example about 20 degrees. In some implementations, the two or more capillaries form respective lumens that are angled with respect to one another at an angle or about 20 degrees. In some implementations, the two or more capillaries form respective lumens that are angled with respect to a longitudinal axis of the catheter body at an angle of about or between 15-25 degrees.

In some implementations, a diameter of at least one of the two or more capillaries and a diameter of at least another one of the two or more capillaries are equal. In some implementations, a diameter of at least one of the two or more capillaries is different from a diameter of at least another one of the two or more capillaries. In some implementations, at least one of the two or more capillaries has an inner diameter that ranges from 25 microns to 400 microns. In some implementations, at least one of the two or more capillaries has an inner diameter that ranges from 40 microns to 100 microns. In some implementations, at least one of the two or more capillaries has an outer diameter that ranges from 30 microns to 1000 microns. In some implementations, at least one of the two or more capillaries has an outer diameter that ranges from 140 microns to 400 microns.

In some implementations, a distance between (i) an intersection of the streams of the gas-enriched liquid and (ii) an end tip of the catheter or an output aperture of a through lumen is greater than or equal to a diameter of the end tip of the catheter. In some implementations, a distance between (i) the first ends of the two or more capillaries and (ii) an end tip of the catheter or an output aperture of a first lumen is greater than or equal to a diameter of the end tip of the catheter.

In some implementations, at least one capillary comprises a fiducial marking.

In some implementations, the catheter body is configured to position the two or more capillaries such that the streams of the gas-enriched liquid intersect and mix with the blood without formation of bubbles.

In some implementations, the catheter body is configured to position the two or more capillaries such that the streams of the gas-enriched liquid minimize outgassing to less than a maximum of 1 milliliter of outgassing of dissolved oxygen from the gas-enriched liquid into the vasculature or tissue.

In some implementations, the catheter body is configured to position the two or more capillaries such that the streams of the gas-enriched liquid minimize outgassing to a maximum of 0.01 milliliters of outgassing of dissolved oxygen from the gas-enriched liquid into the vasculature or tissue.

In some implementations, the streams of the gas-enriched liquid intersect and mix with the blood without formation of bubbles. In some implementations, the streams of the gas-enriched liquid intersect and mix with the blood in a region beyond an end of the catheter body to reduce bubble nucleation along one or more surfaces of the catheter body or at least two capillaries.

In some implementations, the catheter includes one or more sensors, wherein the one or more sensors are configured such that the one or more sensors are in fluid communication with the vasculature of the patient when the catheter is inserted into the vasculature of the patient. In some implementations, at least one of the one or more sensors is an oxygen partial pressure (pO₂) sensor.

In some implementations, the catheter includes a self-centering device configured to center the catheter body within the vasculature of the patient. In some implementations, the self-centering device comprises one or more mesh structures encircling the catheter body. In some implementations, the self-centering device comprises a balloon.

In some implementations, the catheter includes a cover that encloses the catheter body and the two or more capillaries, the cover comprising a soft rubber. In some implementations, the catheter body comprises a polycarbonate material.

In some implementations, the two or more capillaries comprise three capillaries. The catheter includes the catheter body that includes, for each capillary of the three capillaries, a channel in which that capillary is seated, the channel comprising a straight portion and an angled portion, the angled portion causing that capillary to form an angle with respect to a longitudinal axis (or center axis) of the catheter body, the catheter body comprising a flat surface at a distal portion of the catheter body. In some implementations, each capillary is seated in a potting material, the potting material holding that capillary in place with respect to the catheter body. In some implementations, each capillary comprises a glass wall coated in a polyamide coating. In some implementations, for each capillary, the polyamide coating and the glass wall are polished to a flat surface that is flush with the flat surface of the catheter body. In some implementations, the capillaries are each angled at about 10-30° with respect to the center axis. In some implementations, streams of the gas-enriched liquid emitted from each of the capillaries are configured to mix at about 0.5 mm from the flat surface of the catheter body.

In some implementations, a process for forming a catheter for delivering a gas-enriched liquid includes forming a catheter body configured for placement in a vasculature of a patient, the catheter body comprising two or more channels each configured for receiving capillaries that deliver a gas-enriched liquid to the vasculature of the patient when the catheter body is placed in the vasculature of the patient. The process includes coupling two or more capillaries to the catheter body, the two or more capillaries being configured to receive a gas-enriched liquid from a gas-enriched liquid source. The process includes forming an end surface including a first end of the catheter body and first ends of each of the two or more capillaries, the first ends of the two or more capillaries having apertures through which the gas-enriched liquid can flow, the first ends of the two or more capillaries being flush with the first end of the catheter body so that the end surface of the catheter is smooth and includes the apertures for the two or more capillaries. The first ends of the two or more capillaries are positioned relative to one another to cause streams of the gas-enriched liquid to intersect and mix with blood of the patient in a region beyond an end of the catheter body.

In some implementations, a system includes a catheter as described in any of the implementations described in this disclosure. In some implementations, the system further includes a liquid source. In some implementations, the liquid source is a gas-enriched liquid source. In some implementations, the system includes one or more pumps configured to provide the liquid from the liquid source to the two or more capillaries. In some implementations, the system includes one or more pumps configured to provide the gas enriched liquid from the gas-enriched liquid source to the two or more capillaries.

In some implementations, the process includes forming an end surface including a first end of the catheter body and first ends of each of the two or more capillaries. The process includes removing a polyamide coating from the first sends of the two or more capillaries. The process includes polishing the catheter body and the two or more capillaries together to form the end surface that is smooth. In some implementations, the process includes polishing the catheter body and the two or more capillaries together to form the end surface.

In some implementations, the polishing is performed using a flat surface.

In some implementations, the polishing is performed using a curved surface.

In some implementations, the two or more capillaries are configured such that the streams of the gas-enriched liquid minimize outgassing to less than a maximum of 1 milliliter of outgassing of dissolved oxygen from the gas-enriched liquid into the vasculature or tissue.

In some implementations, the two or more capillaries are configured such that the streams of the gas-enriched liquid minimize outgassing to a maximum of 0.01 milliliters of outgassing of dissolved oxygen from the gas-enriched liquid into the vasculature or tissue.

In some implementations, a catheter for delivering a gas-enriched liquid includes a single capillary comprising a lumen formed by a glass wall, the single capillary including a first end, wherein the first end of the single capillary has an aperture through which a gas-enriched liquid can flow. The single capillary is coated in a first coating layer, the first coating layer being adhered to a second coating layer by an adhesive. The first coating layer, the second coating layer, the glass wall, and the adhesive are polished to form a flat, continuous surface comprising the aperture.

In some implementations, the two or more capillaries are configured such that the streams of the gas-enriched liquid minimize outgassing to less than a maximum of 1 milliliter of outgassing of dissolved oxygen from the gas-enriched liquid into the vasculature or tissue.

In some implementations, the two or more capillaries are configured such that the streams of the gas-enriched liquid minimize outgassing to a maximum of 0.01 milliliters of outgassing of dissolved oxygen from the gas-enriched liquid into the vasculature or tissue.

In some implementations, the catheter includes a conical end surface of the catheter and wherein the first ends or apertures of the capillaries are positioned such that the emitted streams are substantially perpendicular relative to the first end of the catheter body.

In some implementations, the catheter comprises a curved, spherical end surface of the catheter and wherein the first ends or apertures of the capillaries are positioned such that the emitted streams are substantially perpendicular relative to the first end of the catheter body.

In some implementations, an outer diameter of a catheter body that supports the single capillary is 0.5 to 3 French.

In some implementations, an outer diameter of a catheter body that supports the single capillary is between 0.5 to 1 French.

In some implementations, the capillary has an inner diameter size of between 30 and 50 microns.

In some implementations, an SSO₂ delivery rate from the capillary is configured to be between 0.25-0.75 ml/min.

In some implementations, the capillary is configured such that the stream of the gas-enriched liquid minimizes outgassing to less than a maximum of 1 milliliters outgassing of dissolved oxygen from the gas-enriched liquid into a vasculature of a patient.

In some implementations, the capillary is configured such that the stream of the gas-enriched liquid minimizes outgassing to a maximum of 0.01 milliliters of outgassing of dissolved oxygen from the gas-enriched liquid into a vasculature of a patient.

In some implementations, the catheter comprises a conical end surface of the catheter and wherein the first ends or apertures of the two or more capillaries are positioned such that the streams from the two or more capillaries are substantially perpendicular relative to the first end of the catheter body.

In some implementations, the catheter comprises a curved, spherical end surface of the catheter and wherein the first ends or apertures of the two or more capillaries are positioned such that the streams from the two or more capillaries are substantially perpendicular relative to the first end of the catheter body.

In some implementations, a diameter of the end surface is between 1 and 6 French.

In some implementations, a diameter of the end surface is between 1 and 3 French.

In some implementations, an inner diameter of at least one of the two or more capillaries is between 15-150 microns.

In some implementations, an outer diameter of at least one of the two or more capillaries is between 50-400 microns.

In some implementations, an inner diameter of at least one of the two or more capillaries is between 40-60 microns.

In some implementations, an SSO₂ delivery rate from the two or more capillaries is between 2-4 ml/min.

In some implementations, a gas-enriched liquid delivery rate from the two or more capillaries is between 2-4 ml/min. In some implementations, a gas-enriched liquid delivery rate from the two or more capillaries is between 0.1-20 ml/min.

In some implementations, an SSO₂ delivery rate from the two or more capillaries is between 0.1-20 ml/min.

In some implementations, an SSO₂ delivery rate from the capillary is configured to be between 0.25-0.75 ml/min.

In some implementations, the catheter body is configured to position the two or more capillaries such that the streams of the gas-enriched liquid minimizes delivery of gas or outgassing to a maximum of 0.01 milliliters of gas or outgassing of dissolved oxygen from the gas-enriched liquid into the vasculature or tissue.

In some implementations, the catheter body is configured to position the two or more capillaries such that the streams of the gas-enriched liquid minimizes delivery of gas or outgassing to a maximum of less than 1 milliliters of gas or outgassing of dissolved oxygen from the gas-enriched liquid into the vasculature or tissue.

In some implementations, the at least two capillaries are configured to deliver the streams of the gas-enriched liquid to deliver oxygen dissolved in liquid at an oxygen concentration of 1 ml/ml to 2 ml/ml.

In some implementations, the streams of the gas-enriched liquid to deliver the oxygen dissolved in liquid with a maximum outgassing of 0.01 milliliters of gas.

In some implementations, the streams of the gas-enriched liquid to deliver the oxygen dissolved in liquid with a maximum outgassing of less than 1 milliliter of gas.

In some implementations, each of the two or more capillaries are in fluid communication with a respective lumen, and wherein each lumen is sized to enable gas-enriched liquid delivery rates between 0.1-20 ml/min total.

In some implementations, a minimum flow rate through each of the two or more capillaries ensures the gas-enriched liquid sprays out of a respective tip for each of the two or more capillaries.

In some implementations, the minimum flow rate enables a Reynolds number of about 1000.

In some implementations, each of the two or more capillaries comprises an inner diameter size of between 25-35 microns and is configured for a delivery rate of 0.25-1.0 ml/min at 37° C. and about 500 pounds per square inch of pressure.

In some implementations, each of the two or more capillaries comprises an inner diameter size of between 45-55 microns and is configured for a delivery rate of 0.4-1.6 ml/min at 37° C. and about 500 pounds per square inch of pressure.

In some implementations, a single capillary comprises an inner diameter size of between 25-35 microns and is configured for a delivery rate of 0.25-1.0 ml/min at 37° C. and about 500 pounds per square inch of pressure.

In some implementations, a single capillary comprises an inner diameter size of between 45-55 microns and is configured for a delivery rate of 0.4-1.6 ml/min at 37° C. and about 500 pounds per square inch of pressure.

In some implementations, the two or more capillaries comprise three capillaries, the three capillaries each having an inner diameter of about 25-35 microns for a total flow rate can be about 0.5 to 3 ml/min.

In some implementations, the two or more capillaries comprise three capillaries, the three capillaries each having an inner diameter of about 45-55 microns for a total flow rate can be about 1 to 5 ml/min.

In some implementations, a system includes a catheter as described in any of the implementations described in this disclosure. In some implementations, the system further includes a liquid source. In some implementations, the liquid source is a gas-enriched liquid source. In some implementations, the system includes one or more pumps configured to provide the liquid from the liquid source to the two or more capillaries. In some implementations, the system includes one or more pumps configured to provide the gas enriched liquid from the gas-enriched liquid source to the single capillary.

In some implementations, there is provided a catheter for insertion into a vasculature of a patient for delivery of a liquid, such as a gas-enriched liquid. The catheter includes a catheter body. The catheter includes two or more capillaries coupled to the catheter body. The two or more capillaries are configured to receive a liquid. An end surface of the catheter includes a first end of the catheter body and first ends of each of the two or more capillaries. The first ends of the two or more capillaries have apertures through which the liquid can flow. The first ends of the two or more capillaries may be flush with the first end of the catheter body. The end surface of the catheter may be smooth or continuous. The end surface of the catheter may include the apertures of the two or more capillaries. The two or more capillaries may be positioned or angled relative to one another to cause streams of liquid or fluid from the two or more capillaries to intersect in a region beyond the first end of the catheter body.

In some implementations, there is provided a method for forming a catheter for. The method includes forming a catheter body for placement in a vasculature of a patient. The catheter body comprises two or more channels each configured for receiving a capillary. The method includes coupling two or more capillaries to the catheter body (within the respective two or more channels). The process includes forming an end surface including a first end of the catheter body and first ends of each of the two or more capillaries. The first ends of the two or more capillaries have apertures. The first ends of the two or more capillaries may be flush with the first end of the catheter body so that the end surface of the catheter is smooth, or continuous, and includes the apertures for the two or more capillaries. The two or more capillaries may be positioned or angled relative to one another to cause streams of liquid or fluid from the two or more capillaries to intersect in a region beyond the first end of the catheter body

In some implementations, there is provided a catheter for delivering a gas-enriched liquid includes a capillary comprising a lumen formed by a glass wall, the capillary including a first end. The first end of the capillary has an aperture through which a liquid can flow. At least a portion of the capillary is coated in a first coating layer. The first coating layer may be adhered to a second coating layer by an adhesive. The first coating layer, the second coating layer, the glass wall, and the adhesive may have a continuous surface comprising the aperture. The surface may be flat. The surface may be a polished surface.

In some implementations, the catheter body is configured to position the two or more capillaries such that the streams of the gas-enriched liquid minimize outgassing with less than a maximum of 0.01 milliliters of gas or outgassing of dissolved oxygen from the gas-enriched liquid into the vasculature or tissue. In some implementations, the catheter body is configured to position the two or more capillaries such that the streams of the gas-enriched liquid minimize outgassing with a maximum of 0.01 milliliters of outgassing of dissolved oxygen from the gas-enriched liquid into the vasculature or tissue.

In general, an implementation described with respect to one aspect may be provided in combination with another aspect. The details of one or more embodiments are set forth in the accompanying drawings and the description. Other features and advantages will be apparent from the description and drawings, and from the claims.

### BRIEF DESCRIPTION OF DRAWINGS

FIG. 1A is a diagram of an example system for enriching a bodily liquid with a gas-enriched liquid inside of an enclosed area of a body.

FIG. 1B shows a portion of an example catheter of the system of FIG. 1A.

FIG. 1C shows an example of a portion an example catheter of the system of FIG. 1A.

FIG. 1D shows a diagram of an example system for enriching a bodily liquid with a gas-enriched liquid inside of an enclosed area of a body.

FIG. 2 is a diagram of an example system for enriching a bodily liquid with a gas-enriched liquid inside of an enclosed area of a body.

FIG. 3 is a perspective view diagram of an example catheter.

FIG. 4 is a side-view diagram of an example catheter.

FIG. 5 is a perspective diagram of an example catheter including a single capillary.

FIG. 6 is a perspective diagram of an example catheter with an overmold material.

### DETAILED DESCRIPTION

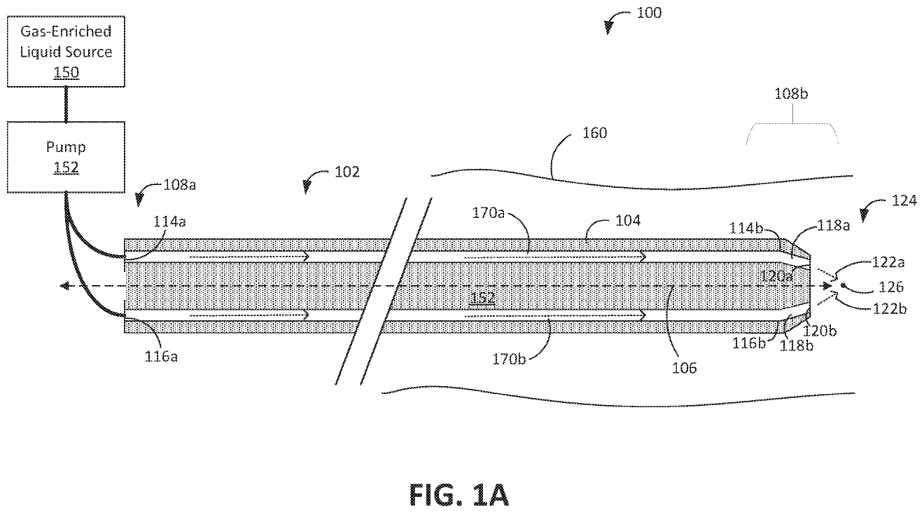

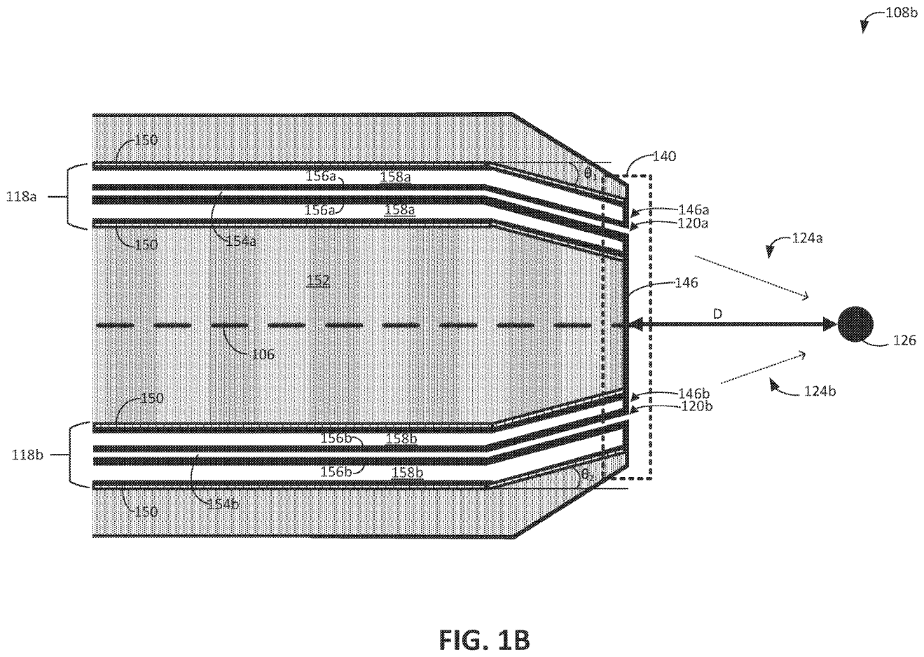

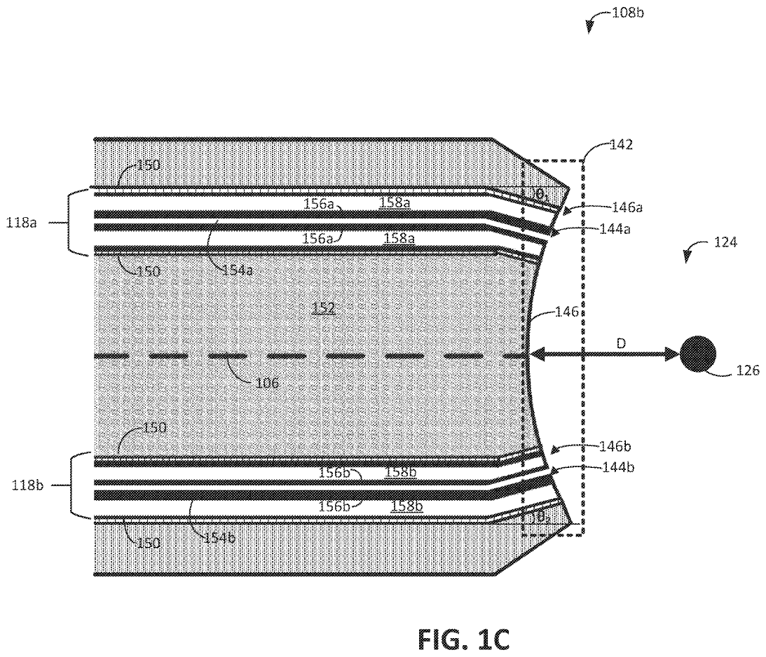

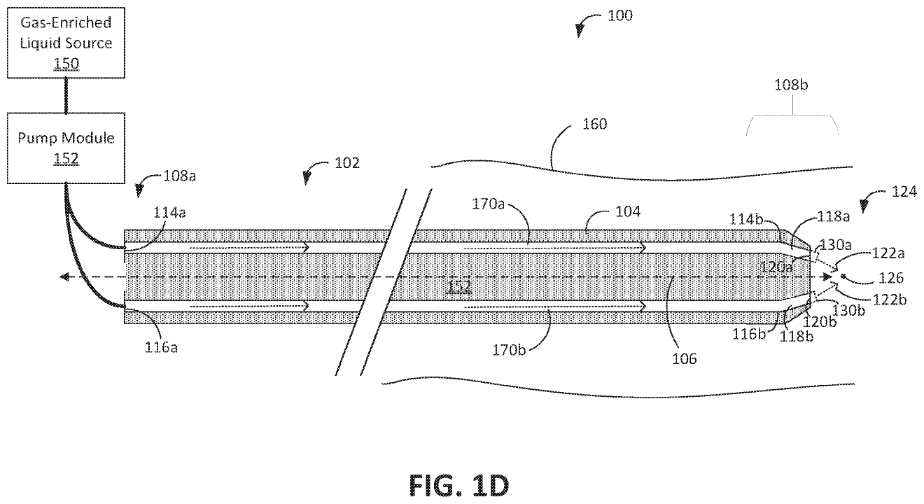

FIG. 1A shows an example system 100 for enriching a bodily liquid with a dissolved gas or gas-enriched liquid inside of an enclosed area of a body. As an example, the system 100 can be used to enrich a patient's blood with supersaturated oxygen enriched liquid or supersaturated liquid within the vasculature of the patient thereby delivering supersaturated oxygen (SSO₂) therapy to a patient, increasing oxygen in the blood and diffusion of oxygen into tissue. In certain implementations, the system 100 with catheter 102 may be configured to deliver gas-enriched liquid, e.g., oxygen enriched liquid or solution, e.g., supersaturated oxygen liquid or solution, which may include liquid having a dissolved O₂ concentration of 0.1 ml O₂/ml liquid (STP) or greater or 0.1-6 ml O₂/ml liquid (STP) or 0.2-3 ml O₂/ml liquid (STP) (e.g., without clinically significant outgassing or gas emboli). When such supersaturated oxygen liquid or solution is mixed with blood, the resulting blood may be referred to as supersaturated oxygen enriched blood. In certain implementations, the resulting blood may have an elevated pO₂ in a target range of 400 mmHg or greater or 600-1500 mmHg or 760-1200 mmHg or around 1000 mmHg.

In one example, the resulting supersaturated oxygen enriched blood may have a pO₂ of 760-1500 mmHg when a patient's blood has a minimum pO₂ of 80 mmHg, their blood flow rate is 50-150 ml/min, the SSO₂ saline flow rate is 2-5 ml/min and the dissolved O₂ concentration in saline is 0.2-3 ml O₂/ml saline (STP). In another example, where the patient's blood is below 80 mmHg, the treatment objective may be to boost the blood pO₂ to above 80 mmHg, so the system may deliver an infusion of supersaturated oxygen enriched liquid that causes the blood to have a pO2 level of 80 mmHg or greater or 80-760 mmHg.

The catheter 102 is configured to deliver gas-enriched liquid, e.g., oxygen-enriched liquid, without clinically significant outgassing or gas emboli. For example, 1.0 ml or more of gas or outgassing of the dissolved oxygen from the gas-enriched liquid in a patient's vasculature or tissue may be considered a clinically significant amount of gas or outgassing of the dissolved gas from the gas-enriched liquid. In certain implementations, the catheter may deliver a stream of gas-enriched liquid (e.g., oxygen-enriched) with no more than 0.99 ml of gas or outgassing of the dissolved gas from the gas-enriched liquid, e.g., about 0.001 to 0.99 ml of gas or outgassing of the dissolved gas from the gas-enriched liquid, in the stream or forming in the vasculature. In certain implementations, the catheter 102 may deliver a stream of gas-enriched (e.g., oxygen-enriched) liquid with less than 0.01 ml of gas or outgassing of the dissolved gas from the gas-enriched liquid, e.g., about 0.001 to 0.009 ml of gas or outgassing of the dissolved gas from the gas-enriched liquid, in the stream or forming in the vasculature. In certain implementations, the system or catheter may include a bubble detector which may initiate a shutdown of the infusion of oxygen enriched liquid if an accumulated volume of 10 microliters of gas or outgassing of the dissolved gas from the gas-enriched liquid (or 0.01 ml) is detected. For example, if 0.01 ml of gas or outgassing of the dissolved gas from the gas-enriched liquid is detected during an infusion lasting up to 120 mins in duration or about 60-120 min, shutdown may be initiated.

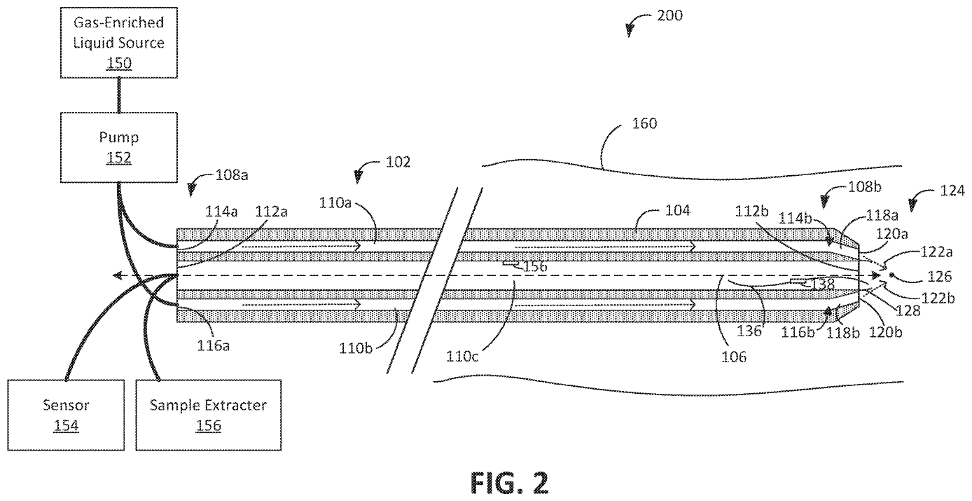

As shown in FIG. 1A, the system 100 includes a catheter 102, a gas-enriched liquid source 151, and a pump 152. The catheter 102 is configured to be inserted into the vasculature of a patient to facilitate the delivery of a gas-enriched liquid (e.g., from the gas-enriched liquid source 151) into the vasculature of the patient via the pump 152. In certain embodiments, the catheter 102 is configured to facilitate the measurement of one or more properties of the patient's blood within the patient's vasculature (e.g., by providing a sensor 154 access to the patient's vasculature) and/or to facilitate the collection of blood samples from the patient's vasculature (e.g., by providing a sample extraction device 156 access to the patient's vasculature), as shown in FIG. 2. For example, in certain embodiments, such as FIG. 2, the sensor 154 may positioned on the distal (or first) end 108b of the catheter 102 or in a communicating lumen 110c of the catheter 102. The sensor 154 can detect various blood parameters (e.g., partial pressure of oxygen in the patient's blood (pO₂), the oxygen saturation of the patient's blood (SO₂), the flow rate of the patient's blood, a temperature of the patient's blood), during treatment or after treatment is paused or completed. As subsequently described in relation to FIG. 1B and FIG. 1C, the catheter 102 includes a distal end 108b (that has a smooth, continuous end surface 140 (or 142). The distal end 108b includes a portion of the catheter 102 where one or more capillaries 118 are configured to emit a stream of the gas-enriched liquid into the vasculature of the patient. The distal end 108b of the catheter 102 is polished smooth to create an end surface where the one or more capillaries 118 are flush with the distal end 108b of the catheter 102. The smooth surface enables a user to position the catheter 102 at more locations within the vasculature of the patient without injuring the patient or damaging the vasculature.

The catheter 102 includes an elongated catheter body 104 (e.g., extending along a longitudinal axis 106 through the center of the catheter body 104) having a proximal (or second) end 108a opposing the distal end 108b. In certain embodiments, the catheter can have a circular, elliptical, or ovular cross-section along a portion of or an entirety of its length. In certain embodiments, the catheter body 104 can be flexible (e.g., such that it can be bent or curved at one or more locations along its length. In certain embodiments, at least a portion of the catheter 102 and/or the catheter body 104 can be composed of polycarbonate, glass, ceramic, stainless steel, polyether ether ketone (PEEK), polyether block amide (PEBA) (e.g., PEBAX produced by Akrema S.A., Colombes, France), acrylonitrile butadiene styrene (ABS), polyimide, and/or other suitable materials. In certain embodiments, the catheter body 104 can have an outer diameter ranging from 2 F to 12 F, or for example, 4 F to 6 F (according to the French scale-about 1.33 mm to 4 mm or about 1.33 mm to 2 mm). In some implementations, the catheter body 104 can have an outer diameter that is about 1 F. In some implementations, the catheter body 104 can have an outer diameter that is between about 1 F to about 14 F. In some implementations, the catheter body 104 can have an outer diameter that is 2 F. In some implementations, the catheter body 104 can have an outer diameter that is 1 F.

Turning briefly to FIG. 2, the catheter 102 can include one or more communicating lumens 110c extending through a center of the catheter body 104 (e.g., along the longitudinal axis 106). In certain embodiments, at least two additional lumens 110a and 110b may extend through opposing sides of the catheter body 104 to connect the capillaries 118a-b to the gas-enriched liquid source 151. The one or more communicating lumens 110c can extend through a center of the catheter body 104 (e.g., along the longitudinal axis 106). The lumens 110a and 110b may be parallel to the communicating lumen 110c. Each of the lumens 110a-110c can have a circular, elliptical, or ovular cross-section along a portion of or an entirety of the lumen length. In certain embodiments, the communicating lumen 110c can have an inner diameter ranging from 0.020 inches to 0.045 inches (about 0.5 mm to about 1.1 mm). Each of the lumens 110a-110c includes a respective input aperture and a respective output aperture. For example, the communicating lumen 110c includes an input aperture 112a on the first (or proximal) end 108a of the catheter body 104 and an output aperture 112b on the second (or distal) end 108b of the catheter body 104. As another example, the lumen 110a includes an input aperture 114a on the first end 108a of the catheter body 104 and an output aperture 114b on the second end 108b of the catheter body 104. As another example, the lumen 110b includes an input aperture 116a on the first end 108a of the catheter body 104 and an output aperture 116b on the second end 108b of the catheter body 104. Returning to FIG. 1A, the catheter 102 includes a capillary 118a extending through channel 170a of the catheter body 152, such that the capillary is seated in the channel in potting material or affixed by an adhesive (e.g., material 150). The capillary 118a terminates at an output aperture 120a. In certain embodiments, the capillary 118a can have an inner diameter between 40 microns and 100 microns. In certain embodiments, the capillary 118a can have an outer diameter between 140 microns and 160 microns. In certain embodiments, the capillary 118a can have a length ranging from 5 cm to 10 cm or be a length “l” which is substantially equal to the diameter of the catheter tip or distal end.

The catheter 102 also includes a capillary 118b extending through the channel 170b of the catheter body 152, such that the capillary is seated in the channel in potting material or affixed by an adhesive (e.g., material 150). The capillary 118b terminates at an output aperture 120b. In certain embodiments, the capillary 118b can have an inner diameter from 40 microns to 100 microns. In certain embodiments, the capillary 118b can have an outer diameter from 140 microns to 400 microns. In certain embodiments, the capillary 118b can have a length ranging from 5 cm to 10 cm or be a length “l” which is substantially equal to the diameter of the catheter tip or distal end. Capillary 118b can be identical to the capillary 118a or can have a different diameter and/or angle with respect to the axis 106 than capillary 118a. For example, in certain embodiments, the capillaries 118a and 118b may have identical or different sized inner and/or outer diameters. In certain embodiments, the capillaries 118a and 118b may have identical or different sized lengths.

In certain embodiments, there are more than two capillaries 118a-b. For example, the catheter 102 can include three capillaries, four capillaries, five capillaries, and so forth. Each capillary 118a-b (or 1181-n, where n is a number greater than two) is seated or positioned in a respective channel 170a-b (or 1701-n, where n is a number greater than 2). Each channel 170a-b is made in the catheter body 152 to guide a capillary, at the distal end 108b, to an angle θ with respect to axis 106 for mixing streams of the gas-enriched liquid. The channels 170a-b position the capillaries 118a-b within the catheter body 152 for a length of the catheter body. Generally, the channels 170a-n are parallel with one another and axis 106 along a length of the catheter body 152, except at the distal end 108b in which the channels form angles with respect to the axis 106. In certain embodiments, the capillaries 118a-b are placed in the respective channels 170a-b, and a potting material 150 is used to couple the capillaries 118a-b to the catheter body 152. Once the capillaries 118a-b are bonded to the catheter body 152, the distal end 108b is polished to form an end surface 140 or 142, described in relation to FIGS. 1B and 1C.

In certain embodiments, either or both of the capillaries 118a-b may have respective inner diameters ranging from 15-150 microns. In certain embodiments, either or both of the capillaries 118a-b can have respective outer diameters ranging from 50-400 microns. In certain embodiments, either or both of the capillaries 118a-b can have lumens with inner diameters that are sized independently from one another in the range of 15-150 microns. In certain embodiments, either or both of the capillaries 118a-b can have lumens with outer diameters that are sized independently from one another with the range of 50-400 microns.

In certain embodiments, lumens of the capillaries 118a-b can be sized to enable different gas-enriched liquid, e.g., SSO₂, delivery rates. For example, the lumens can be sized to enable gas-enriched liquid delivery rates between 0.1-20 ml/min (total). The flow rate through the capillary is a factor of the capillary diameter as well as length. By lengthening or shortening the capillary, the amount of fluid friction can be varied and thereby the flow rate varied. The minimum flow rate that ensures the fluid will spray out of the tip and not drip, thereby ensuring fluid mixing. The Reynolds number takes fluid velocity into account. The maximum flow rate is based on Re=1000, which is the end of laminar flow in a circular conduit.

For example, for a single lumen capillary having an inner diameter size of about 30 microns, the delivery rate can be 0.25-1.0 ml/min under ideal conditions (37° C., 500psi). For example, for a single lumen capillary having an inner diameter size of about 50 microns, the delivery rate can be 0.4-1.6 ml/min under ideal conditions (37° C., 500 PSI). For example, for a catheter 102 having three capillaries configured to deliver gas-enriched liquid when each lumen of a capillary has an inner diameter of about 30 microns, a total flow rate can be about 0.5 to 3 ml/min. For example, for a catheter 102 having three capillaries configured to deliver gas-enriched liquid when each lumen of a capillary has an inner diameter of about 50 microns, a total flow rate can be about 1-5 ml/min.

During an example usage of the system 100, the gas-enriched liquid source 151 and the pump 152 are coupled to the catheter 102, such that the gas-enriched liquid source 151 and the pump 152 are in fluid communication with the input apertures 114a and 116a of the lumens 110a and 110b and/or capillaries 118a and 118b, respectively. As an example, one or more fluid-tight tubes can be used to convey gas-enriched liquid from the gas-enriched liquid source 150 to the pump 152, and from the pump 152 to the input apertures 114a and 116b. In certain embodiments, one or more fluid-tight tubes can be used to convey gas-enriched liquid from the gas-enriched liquid source 150 to the input apertures 114a and 116b, where at least a portion of the one or more fluid-tight tubes are coupled to a peristaltic pump or form part of the peristaltic pump, which urges fluid from the gas-enriched liquid source to the input apertures 114a and 116b. In certain embodiments, the tubes can be secured to the input apertures 114a and 116b using a fitting or connector, such as a high-pressure Luer fitting.

In certain embodiments, the gas-enriched liquid source 150 can include one or more storage tanks for storing the gas-enriched liquid. In certain embodiments, the gas-enriched liquid can be a supersaturated oxygen enriched liquid or supersaturated liquid, such as a liquid having a dissolved oxygen (O₂) concentration between 0.2 and 3 ml O₂/ml solvent (which is the concentration equivalent of 100 psi to 1500 psi). In certain embodiments, the gas-enriched liquid can include liquid enriched with oxygen, ozone, inert gas, nitrogen, nitrous oxide, carbon dioxide, and/or air. In certain embodiments, the gas-enriched liquid source 150 may include an oxygenation device, which is operated by a console or hardware component that controls operation of the oxygenation device, as described in U.S. Pat. No. 9,919,276, the entire disclosure of such patent being expressly incorporated herein by reference. The console or hardware component may include a controller, processor, memory and associated circuitry. The oxygenation device may include a fluid supply chamber for receiving a physiologic liquid e.g., saline from an IV bag, and an atomization chamber for receiving a suitable gas, e.g., oxygen from an oxygen tank. The saline is pumped into the oxygen-pressurized atomization chamber and atomized to create gas-enriched or supersaturated liquid, e.g., supersaturated oxygen-enriched saline or supersaturated saline. In certain implementations, the gas-enriched liquid can be oxygen enriched liquid or solution, e.g., supersaturated oxygen enriched liquid or solution, may include liquid having a dissolved O₂ concentration of 0.1 ml O₂/ml liquid (STP) or greater or 0.1-6 ml O₂/ml liquid (STP) or 0.2-3 ml O₂/ml liquid (STP) (e.g., without clinically significant gas emboli). In certain embodiments, the gas-enriched liquid can be a supersaturated oxygen enriched liquid or solution (e.g., saline with a dissolved O₂ concentration in saline of 0.1 ml O₂/ml saline (STP) or greater or 0.1-6 ml O₂/ml saline (STP) or 0.2-3 ml O₂/ml saline (STP) (e.g., without clinically significant gas emboli).

In certain embodiments, the catheter 102 is configured to deliver oxygen dissolved in a liquid or solution. The catheter 102 can deliver the oxygen dissolved in the liquid at about 500-1000 pounds per square inch (PSI). In an example, the catheter 102 delivers about 0.2-3 ml O₂/ml liquid at 100-1500 PSI to prevent outgassing. In another example, the catheter 102 delivers gas-enriched liquid at 500-1000 PSI with an oxygen concentration between 1 ml O₂/1ml liquid to 2 ml O₂/1ml liquid. In some embodiments, the catheter 102 minimizes outgassing, as previously described. While outgassing can be limited to 0 ml, a maximum amount of outgassing can be no more than 0.99 ml of oxygen out of liquid when the oxygen-enriched liquid is delivered to tissue. In another embodiment, the catheter 102 limits outgassing to no more than 0.01 ml of oxygen out of liquid when the oxygen-enriched liquid is delivered into the blood stream.

Further, a portion of the catheter 102 is inserted into a patient, such as the second end 108b of the catheter body 104 and is positioned within a vasculature of a patient (e.g., a blood vessel 160, such as a vein or artery). After the catheter 102 has been inserted into the patient, the pump 152 is activated, such that it draws the gas-enriched liquid from the gas-enriched liquid source 150, and pumps the gas-enriched liquid, e.g., supersaturated liquid, into each of the lumens 110b and 110c. The gas-enriched liquid flows through the lumens 110b and 110c and into the capillaries 118a and 118b and is expelled from the output apertures 120a and 120b as two respective streams 122a and 122b.

In certain embodiments, the system 100 can be configured to expel streams according to different flow rates and/or pressures. For example, the system 100 can be configured to expel streams between 1 mL/minute (e.g., at a pressure of 100 psi, about 690 kPa) to 3 mL/minute (e.g., at a pressure of 300 psi, about 2 MPa).

The capillaries 118a and 118b are configured such that the streams 122a and 122b intersect with one another and mix in a mixing region 124 within the vasculature of the patient. For example, the capillaries 118a and 118b can define respective paths that are angled relative to the longitudinal axis 106, such that the streams 122a and 122b are expelled from the output apertures 120a and 120b at respective angles relative to the longitudinal axis 106. In certain embodiments, the capillaries 120a and 120b can be configured such that the streams 122a and 122b intersect at a point 126 beyond the tip of the catheter 102 (e.g., where the point 126 is on or around the longitudinal axis 106). For example, the streams 122a and 122b may mix without bubble formation or without significant bubble formation in the mixing region 124 at a distance downstream from the output apertures of the capillaries 118a and 118b.

The distal portion 108b of the catheter 102 includes an end surface 140, shown in FIG. 1B. The capillaries 118a-b are polished flush with the catheter body 152 of the catheter 102 to form a solid continuous surface, except for apertures 120a and 120b of the capillaries 118a-b. For example, as shown in FIG. 1D, respective portions 130a-b of the capillaries 118a-b are removed so that the end surface 140 is smooth.

FIG. 1B shows a view of an example of the distal portion 108b of the catheter 102 of FIG. 1A. The distal portion includes tapered end of a catheter body 152 of the catheter 102. The catheter body 152 includes channels for receiving the capillaries 118a-b. In other words, the capillaries 118a-b are seated in channels in the catheter body 152. The capillaries 118a-b are adhered to the catheter body 152 by an adhesive 150. The adhesive conforms the capillaries 118a-b to follow the channels in the catheter body 152. Walls 156a-b of the respective capillaries 118a-b form lumens 154a-b through which the gas-enriched liquid flows out of the catheter 102. For example, capillary 118a has a wall 156a that is adhered to the catheter body 152 by an adhesive 150. The wall is a semi-rigid material, such as a glass, that can follow the angle θ of the channel in the catheter body 152. The angle θ of each capillary 118a-b enables streams of the gas-enriched liquid emitted by the capillaries to follow path 124a to a mixing zone 126 outside of the catheter 102 and in the vasculature of the patient. Similarly, capillary 118b has wall 156b forming a lumen 154b. The capillary is adhered to the catheter body 152 by adhesive 150. The capillary 118b forms an angle θ that enables a stream of the gas-enriched liquid to flow along path 124b to mixing region 126 at a distance D from the end surface 140 of the catheter 102. In certain embodiments, D is about 0.5 mm. A steeper (larger) angle θ enables smoother mixing and a smaller distance D, but generally requires a larger catheter end surface 140. In certain embodiments, θ is 16.5°.

In some implementations, a largest diameter of the catheter, e.g., the end surface, is between 1 to 6.5 French. In some implementations, a largest diameter of the catheter is between 4-7 French. In some implementations, a largest diameter of the catheter is between 1 to 3 French. In some implementations, a largest diameter of the catheter is between 1 to 7 French.

Generally, the catheter body 152 is formed from a plastic material. For example, the catheter body 152 can be formed from a polycarbonate material. The material is rigid or semi-rigid and is configured to have channels for seating capillaries 118a-b.

The capillaries 118a-b have respective walls 156a-b. Each wall 156a-b may include a glass material. The material may be rigid or semi-rigid. For example, the glass wall 156 of a capillary 118 is configured to bend slightly at angle θ to enable the capillary to emit a stream of gas-enriched liquid at the angle relative to the end surface 140 of the distal end 108b of the catheter 102.

The walls 156 of the capillaries 118 are seated in channels of the catheter body 152. In certain embodiments, the channels in the catheter body 152 are curved at angle θ, and the glass walls 156 of the capillaries 118 are adhered to the channels using an adhesive 150 to follow the curvature or angle. The adhesive 150 is configured to adhere the capillaries 118 to the catheter body 152 so that the capillaries do not move relative to the catheter body. In certain embodiments, the adhesive 150 bonds the coating 158a-b around walls 156a-b of the capillaries 118a-b to the catheter body. In certain embodiments, the adhesive 150 includes a potting compound and forms a thicker layer in the channels of the catheter body 152.

The walls 156 of the capillaries 118 may be coated in a coating 158. For example, capillary 118a includes a coating 158a around wall 156a, and capillary 118b includes a coating 158b around wall 156b. The coating can include a shatter-resistant or shatter-proof material that prevents the glass walls 156a-b of the capillaries 118a-b from breaking into pieces. The coating 158 is a flexible material that enables the glass walls 156 to flex without breaking. In certain embodiments, the coating 158 includes a polyamide material.

In certain embodiments, the capillaries 118a-b form different angles θ₁ and θ₂. The angles θ₁ and θ₂ are selected based on the distance D desired from the end surface 140 of the catheter 102 for the mixing zone 126. In certain implementations, the angles θ₁ and θ₂ are identical. In certain implementations, the angles θ₁ and θ₂ can be 10°, 15°, 20°, 30°, or any value less than or equal to 45°. In certain implementations, the angles θ₁ and θ₂ are selected to accommodate different values of D including 1 mm, 2 mm, or any distance less than 5 mm.

The distance D is a function of the angles θ₁ and θ₂ and the distance of the capillaries from the catheter longitudinal centerline. For example, if a catheter has capillaries positioned such that the angles θ₁ and θ₂ are 16.5°, and the distance of the capillaries from the catheter longitudinal centerline is 0.207 mm, the distance D would be about 0.70 mm. For other sized catheters, where capillaries are be positioned at different angles θ₁ and θ₂, the distance D would vary. The distance between the catheter longitudinal centerline and the capillaries 118a-b may vary, e.g., the distance may be less than 1 mm. The angles for θ₁ and θ₂ may be 5° to 20°. When an angle θ₁ or θ₂ decreases, the value for D will increase. When the distance of the capillaries from the catheter longitudinal centerline decreases, then D will decrease. In certain implementations, a 5 French (1 Fr=0.333mm) catheter having three capillaries may have a focal point for the streams (located a distance D from the end surface of the catheter) with a value of D=0.5 mm depending on the angle and centerline distance.

The end surface 140 is polished to a smooth, flat surface. The end surface 140 is generally continuous, except for apertures 120a and 120b formed from the respective lumens 154a-154b of the capillaries 118a-b. For example, the flat end surface 140 of the distal end 108b of the catheter 102 is formed from the catheter body 152 of the catheter, the wall of 156a of the capillary 118a, the wall 156b of the capillary 118b, the adhesive layers 150 that couple the walls 156a-b to the catheter body 152, and a coating 158a-b over the walls 156a-b of the capillaries 118a-b.

In certain embodiments, the catheter 102 may be formed as now described. The catheter body 152 is obtained and the channels are cut to enable placement of the capillaries 118. The capillaries 118 or catheter body 152 are coated in adhesive 150, and the capillaries 118 are placed within respective channels of the catheter body 152. The capillaries 118 generally extend slightly from the end surface of the catheter body 152.

If the capillaries 118 were to extend from the end surface 140, the coating 158a-b would be peeled back from the ends 146a-b of the walls 156a-b of the capillaries 118a-b. This exposes the glass of the walls 156a-b. The coating is conventionally removed about 1 mm. The coating 158a-b is removed from the glass walls 156a-b of the capillaries because the coating causes blood clotting to occur. However, exposed glass walls 156a-b are fragile and can be sharp, potentially causing injury to the vasculature of the patient.

In this example, the coating 158 and glass walls 156 are polished along with the catheter body 152 to a flat end surface 140 so that no glass is exposed from the distal end 108b of the catheter 102. A plate can be used to polish the end surface 140 of the distal portion 108b of the catheter 102. The ends 146a-b of the capillaries 118a-b form a smooth continuous surface with the catheter body 152. The end surface 140 is smooth to prevent regions of blood clotting or recirculation when the catheter 102 is inside the vasculature of the patient. Recirculation is prevented or minimized by mixing the streams of gas-enriched liquid at a distance D that is a sufficient distance from the end surface 140 thereby reducing or eliminating dead zones that include localized vortices of blood.

FIG. 1C shows a view of an example of the distal portion 108b of the catheter 102 of FIG. 1A. The distal portion 108b of the catheter 102 of FIG. 1B is similar to FIG. 1B except that it includes a curved end surface 142 instead of a flat surface 140. The curved end surface 142 is configured to enable the capillaries 118a-b to emit gas-enriched liquid at streams that intersect at region 126. In this example, the capillaries 118a-b may require less flexing or bending to achieve a desired angle θ for emitting the steam of the gas-enriched liquid. The end surface 142 is formed in a similar manner to the end surface 140, except a curved polishing plate is used instead of a flat polishing plate to achieve a curved or concave end surface. In an embodiment having a curved, spherical end surface 142, the capillary apertures are generally oval in shape, and the capillary ends or apertures are positioned such that the emitted stream is perpendicular to or nearly perpendicular, e.g., substantially perpendicular, relative to the polished end surface of the catheter body 152. In another embodiment, the end surface 142 may be generally conical in shape. In such an embodiment, the capillary apertures are generally circular in shape, and the capillary ends or apertures are positioned such that the emitted stream is perpendicular to or nearly perpendicular, e.g., substantially perpendicular, relative to the polished end surface of the catheter body 152.

As shown in FIG. 2, when the catheter includes a communicating lumen, the communicating lumen 110c provides access to the vasculature of the patient. For example, in certain embodiments, a sensor 154 can be at least partially inserted into the communicating lumen 110c, such that it is in fluid communication with the blood of the patient. In other implementations, the sensor may be located outside of the communicating lumen 110c or on a catheter wall. The sensor 154 can obtain one or more sensor measurements regarding the blood and provide feedback regarding measured parameters affected by the SSO₂ therapy in order to optimize the SSO₂ therapy. For example, the sensor 154 can measure a partial pressure of oxygen of the patient's blood, an oxygen concentration or SO₂ of the patient's blood, a pressure of the patient's blood, e.g., arterial blood pressure, a flow rate of the of the patient's blood, and/or a temperature of the of the patient's blood.

Examples of such sensors include the following:

One example of a sensor for measuring a partial pressure (pO₂) of oxygen or oxygen saturation SO₂ in the patient's blood is a pulse oximeter. A pulse oximeter may be used for estimating arterial pO₂ or SO₂. Pulse oximetry estimates the percentage of oxygen bound to hemoglobin in the blood. A pulse oximeter uses light-emitting diodes and a light-sensitive sensor to measure the absorption of red and infrared light. In another example, a sensor for measuring partial pressure of oxygen comprises an electrode such as a Clark electrode for measuring pO₂. A Clark electrode is an electrode that measures ambient oxygen concentration in a liquid using a catalytic platinum surface according to the net reaction O₂+4e−+4H+→2H2O. The various sensors may be coupled to a controller of the system via a cable or other wired connection or via a wireless connection.

The processor can receive the signals from these sensors, which signals correspond to the measured values of pO₂. The processor compares the measured pO₂ to a target range of blood pO₂, e.g., 760-1500 mmHg (about 100 kPa to 200 kPa). The target range may be calculated based on a blood flow rate of 50 -150 ml/min, saline flow rate of 2-5 ml/min and dissolved O₂ concentration in saline of 0.2-3 ml O₂/ml saline (STP). The controller can adjust the saline flow rate and/or dissolved O₂ concentration in saline based on the measured pO₂ in blood to achieve an arterial blood pO₂ within the target range. The processor may generate an alert, e.g., through a user interface, audible alarm and/or visual alarm that indicates the level of pO₂. The measured pO₂ indicates the effectiveness of the supersaturated oxygen therapy, letting the caregiver know if the pO₂ in blood is within the target range for optimizing the delivery of oxygen to the patient's ischemic tissue. In certain implementations, the processor may control the delivery of supersaturated oxygen therapy by modifying one or more of the above referenced saline or oxygen parameters based on the signals received from the sensors.

Another example of a sensor is an O₂ fluorescence probe. The fluorescence probe may be coupled to a controller of the system via a cable or other wired or wireless connection. A light source of the O₂ fluorescence probe is illuminated. A fiber optic cable can be used to provide light to the light source in certain implementations, where the fiber optic cable is connected to the controller of the system. The fluorescence of a sensor molecule of the O₂ fluorescence probe is measured. The sensor molecule can include fluorophore. A signal is received by the processor from the O₂ fluorescence probe based on the fluorescence measurement. Fluorescence is measured by measuring the lifetime or decay of the fluorescence intensity signal from the illuminated sensor molecule (e.g., fluorophore) on the fluorescence probe. The decay of this signal is caused by the quenching effect of oxygen molecules in the blood or in tissue on the fluorescence intensity signal of the sensor molecule. The processor can determine the oxygen concentration, SO₂ or pO₂ in blood or tissue based on the quenching effect of oxygen on the florescence intensity signal of the florescence probe. Changes in the amount of time that is required for the signal to decay due to oxygen quenching are indicative of the local oxygen concentration, SO₂ or pO₂ in blood or tissue. The processor generates an alert, e.g., through a user interface, audible alarm and/or visual alarm, based on the determined oxygen concentration, SO₂ or pO₂ in blood or tissue. The alert may indicate the effectiveness of the supersaturated oxygen therapy. The determined oxygen concentration, SO₂ or pO₂ indicates the effectiveness of the supersaturated oxygen therapy, letting the caregiver know if the oxygen concentration, SO₂ or pO₂ in blood is within a predefined target range (e.g., the expected range for a healthy individual) for optimizing the delivery of oxygen to the patient. In certain implementations, the processor may control the delivery of supersaturated oxygen therapy by modifying one or more of the saline or oxygen parameters, e.g., saline flow rate or dissolved O2 concentration in saline, based on the determined oxygen concentration, SO₂ or pO₂ values.

Another example of a sensor is a temperature sensor located on or in the catheter. For example, a thermistor may be utilized to measure the blood temperature of the patient. The processor can receive signals from the thermistor, which signals correspond to the measured values of the blood temperature. The processor may generate an alert, e.g., through a user interface, audible alarm and/or visual alarm that indicates the blood temperature, which may alert the caregiver of a hypothermic or hyperthermic, e.g., febrile, state of the patient.

An example sensor for measuring an arterial pressure of the patient's blood would be a pressure sensor positioned in or coupled to the communicating lumen 110c. The communicating lumen 110c may be used for direct measurement of arterial pressure. The communicating lumen 110c may be connected to a fluid-filled system, which is connected to an electronic pressure transducer. A change in detected blood pressure may be indicative of improved perfusion and/or restored flow in ischemic tissue as a result of the SSO₂ therapy. The therapy may result in improved heart function. In certain implementations, the processor may control the delivery of supersaturated oxygen therapy based on the arterial pressure feedback.

An example sensor used to determine a blood flow rate includes a temperature sensor, e.g., a thermistor, thermocouple or thermal anemometer. A temperature sensor may be located on a catheter tip, capillary tip or in the communication lumen. The temperature sensor may be heated, such that the sensor temperature is raised. As blood flows past the temperature sensor, the degree to which the temperature sensor cools down is indicative of the flow rate past the temperature sensor. The determined blood flow rate may be fed back to the system and may be indicative of the efficacy of the SSO₂ therapy, which results in improved perfusion and/or restored flow in ischemic tissue. In certain implementations, the processor may control the delivery of supersaturated oxygen therapy based on the blood flow rate feedback.

If the sensor is a pressure sensor, the sensor may detect a pressure differential between ambient pressure and arterial pressure or an absolute value of arterial pressure. The pressure sensor may be placed anywhere in the communicating lumen but does not necessarily have to be positioned in the communicating lumen and can be located outside of the lumen. One example of a pressure sensor is a strain gauge. In a catheter having multiple communicating lumens, a pressure sensor may be located in a first communicating lumen providing an uninterrupted pressure signal while blood sampling may be performed via a second communicating lumen simultaneously. In another example, two pressure sensors can be used, with one located in a first communicating lumen and one located in a second communicating lumen to provide redundancy of pressure readings.

In another example, the catheter 102 includes a wire 136 that extends from a distal end 108b of the catheter 102 into the vasculature 160 of the patient. The wire 136 supports sensors. Sensors can be instances of a same type of sensor or different types of sensors. In an example, each of sensors (such as sensor(s) 138) includes a pressure sensor configured to measure blood pressure in the vasculature 160 of the patient. The two pressure sensors can provide a pressure differential (Δp) value for a region of the vasculature around infarct areas, e.g., the sensors may record a blood pressure drop across the infarct areas. In certain embodiments, the sensor(s) 138 are configured for measuring a relative difference in blood flow in the myocardium of the patient. When using wire-based IMR to estimate microvascular resistance, blood flow data is derived via the thermodilution method. Temperature sensors (e.g., thermistors) located at the proximal and distal end of the wire may be utilized to measure the change in cold saline temperature. For example, a pressure sensor on the distal end of the wire can act as a distal thermistor, while a pressure sensor on the proximal shaft of the wire serves as a proximal thermistor. Accordingly, a mean transit time (Tₘₙ) of room-temperature saline injected into a coronary artery can be determined from a thermodilution curve. Using the known thermodilution technique, a correlation between the inverse of Tₘₙ (1/Tₘₙ) and absolute coronary flow is shown. Absolute coronary flow≈1/Tₘₙ·IMR=Δp/(1/Tmn). In general, IMR=pressure/flow.

In certain implementations, IMR (index of microcirculatory resistance) may be calculated using the mean distal coronary pressure Pd and myocardial flow, which is equal to 1/Tₘₙ, where Tₘₙ is mean transit time. The minimal achievable resistance is calculated by making the measurement during maximal hyperemia. Accordingly, IMR=Pd/(1/Tmn) or Pd*Tmn at maximal hyperemia. IMR can provide qualitative information about resistance to flow in the vasculature (e.g., regional microvasculature) after inducing hyperemia. A change in IMR over time in the direction of normal IMR would be a desirable result and a measurable effect of gas-enrichment or SSO₂ therapy.

In certain embodiments, the sensors 138 include flow sensors configured to measure the flow of blood directly in the patient. The flow sensors can include electromagnetic, mechanical, or ultrasonic flow sensors.

The sensors 138 may send pressure measurements (or measurements of another physiological parameter) to a controller (not shown) for estimating the microvascular resistance in the microvascular region. The estimate can include determining a ratio of a change in pressure to the flow. In certain embodiments, at least one sensor 138 includes a pressure sensor. The physiological parameter includes a pressure or pressure change of blood in the vasculature 160 of the patient. In certain embodiments, at least one of the sensors 138 includes a flow sensor. The physiological parameter thus includes a flow rate of blood in the vasculature of the patient.

In certain embodiments, the wire 136 is positioned on or near a distal end 108b of a delivery catheter 102. In certain embodiments, the one or more sensors 138 are positioned directly on the body of the catheter 152. The wire 136 can extend through a communicating lumen 110c of the catheter 102 out of the distal end 108b of the catheter. In certain embodiments, the wire 136 extends along the catheter shaft or on an exterior of a catheter lumen 110c.

In certain embodiments, the wire 136 is positioned on a separate probe that is not directly attached to a delivery catheter 102. In certain embodiments, a second, separate catheter is used to support the one or more sensors 138. In such an example, the catheter 102 is not the delivery catheter, but a second, different catheter, and the delivery catheter is not shown.

As another example, in certain embodiments, a sample extraction device 156 can be used to obtain a sample of the patient's blood via the communicating lumen 110c. For example, the sample extraction device 156 can include one or more pumps or syringes to draw a sample of the patient's blood through the communicating lumen 110c and out of the patient's body. The syringe may be coupled to a proximal end of the catheter for sampling. A valve or stopcock may be included at the proximal end of one more lumen of the catheter to control sampling.

In some implementation, the communicating lumen 110c can also be used to guide the catheter 102 within the patient's body. For example, a guide wire can be inserted into the communicating lumen 110c and manipulated to control the shape and/or position of the catheter 102 within the patient's body.

Further, the catheter 102 may be configured in such a way that eliminates or otherwise reduces the formation of bubbles within the vasculature of the patient. For example, the streams 122a and 122b mix in a mixing region 124 away from any surfaces of the catheter 102 or capillaries thereby reducing, preventing or reducing the likelihood of bubble formation through nucleation on the surfaces of the catheter 102 or capillaries. Preventing or reducing bubble formation includes reducing nucleation of the blood to below a clinically significant threshold.

In certain embodiments, the output aperture 112b of the communicating lumen 110c may be recessed relative to the output apertures 120a and 120b of the capillaries 118a and 118b (e.g., by a distance d along a direction of the longitudinal axis 106). Accordingly, the streams 122a and 122b are less likely to impinge on the surfaces of the lumen 110c, thereby further preventing or reducing the likelihood of bubble formation through nucleation (e.g., such that nucleation, if it occurs, does not have clinically significant gas emboli). Further, the streams 122a and 122b are less likely to interfere with the obtaining of sensor measurements by the sensor 154. In certain embodiments, the distance d can be selected based on the dimensions of the tip of the catheter 102. For example, the distance d can be at least as long as the diameter of the tip or distal end of the catheter 102. In certain embodiments, the distance d can be 0.060 inches (about 1.5 mm) to 0.100 inches (about 2.5 mm). In certain embodiments, the catheter 102 can also include one or more shields or guards 128 (e.g., protrusions, walls, bumps, etc.) positioned over the output aperture 112b to reduce or eliminate nucleation or recirculation near or along one or more surfaces of the catheter.

In certain examples, d is scaled to the diameter of the catheter. The diameter of the catheter tip or distal end will vary depending on factors such as artery size, blood flow rate, and blood back flow. The mixing region, where the streams intersect, may create a mixing zone away from the catheter distal end. Because the diameter of catheter tip will affect blood flow and turbulence in that mixing zone, it is important to ensure that the mixing zone is at least a distance D from the catheter tip and the output aperture of the communicating lumen 110c. In certain examples, a distance d that is substantially equal to the diameter of the catheter tip is the minimum distance that the mixing zone should be located from the catheter tip. This helps avoid the effects of the catheter tip diameter on mixing. A recessed output aperture of the communicating lumen 110c also helps ensure the reliability of blood samples removed via the communicating lumen 110c. It is desirable for the mixing zone to be at least a distance D away from the output aperture of the communicating lumen 110c, to avoid sampling of unmixed SSO₂ solution. Also, when detecting pressure, a sufficiently recessed output aperture of a communicating lumen 110c may result in a reduction in noise in the pressure measurement. In certain embodiments, the distance D can be 0.060 inches (about 1.5 mm) to 0.100 inches (about 2.5 mm). Further, one or more of the surfaces of the catheter 102 (e.g., the interior surfaces of the lumens 110a-110b, the interior surfaces of the capillaries 118a and 118b or capillary walls, the exterior surfaces of the capillaries 118a and 118b, the exterior surfaces of the catheter body 104, etc.) can be smooth to prevent or further reduce the likelihood of bubble formation through nucleation (e.g., such that any gas emboli, if present, are not clinically significant). In certain embodiments, one or more of these surfaces can be coated or pre-wetted with liquids and/or hydrophilic agents or coatings (e.g., saline, ethanol, benzalkonium heparin, blood proteins, etc.) before use to further inhibit the formation of bubbles through nucleation.

In certain embodiments, a target flow rate is about 4.0 ml/min at 500 psi. In certain embodiments, an angle of the three jets from a center line is about 16.5°. In certain embodiments, a distal end 108b maximum diameter is about 1.15 mm or 3.5 French. In certain embodiments, the t capillaries are about 24 mm long. In certain embodiments, the capillaries have an inner diameter of about 50 μm and an outer diameter of about 150 μm. In certain embodiments, the distal end surface 140 or 142 is bonded with epoxy and polished, and no glass or capillary extends beyond distal end surface. A targeted flow rate is 4.0 ml/min at 500 psi.

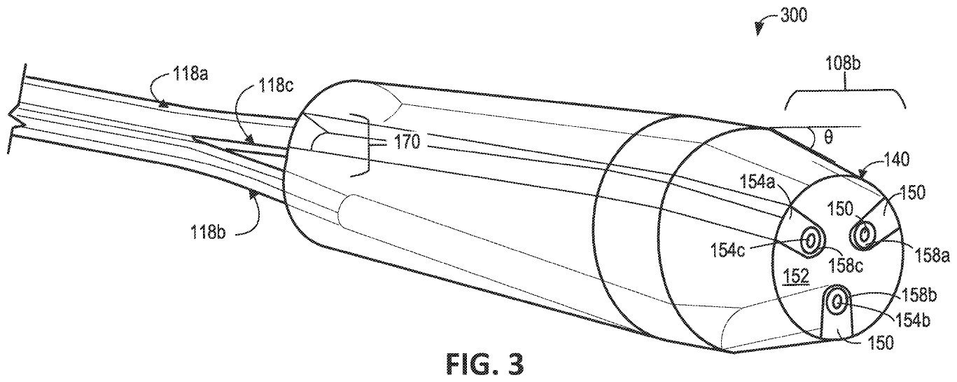

As shown in FIG. 2, communicating lumen 110c extends along a longitudinal axis 106 through a center of the catheter body 104. However, this need not always be the case. Further, in the example shown in FIG. 2, the catheter 102 includes two lumens 110a and 110b that can connect respectively to capillaries 118a and 118b for conveying a gas-enriched liquid into the vasculature of a patient. However, this also need not always be the case. FIGS. 3-6 show alternative embodiments of other example catheters. In these examples, the catheters can include three capillaries or a single capillary extending through the catheter body, each configured to convey a gas-enriched liquid into the vasculature of a patient, as subsequently described.

In certain embodiments, the system 100 can also include self-centering mechanisms for positioning the catheter 102 within the vasculature of the patient (e.g., such that the catheter body 104, capillaries and/or the streams expelled by the capillaries are positioned centrally in a vein or artery, away from the vessel walls to reduce or prevent nucleation or bubble formation (e.g., without clinically significant gas emboli) by preventing the streams from hitting a vessel wall). For example, example catheter 102 may include an inflatable balloon structure. The balloon structure encircles a portion of the catheter 104 and can be selectively inflated and deflated. For example, after a portion of the catheter 102 has been inserted into a vessel 160, the balloon structure can be inflated such that it contacts the walls of the vessel 160. In this configuration, the catheter 104 body is positioned centrally within the vessel 160. Accordingly, the streams of gas-enriched liquid expelled by the capillaries 118a and 118b mix in a mixing region away from the walls of the vessel 160. This can be beneficial, for example, in reducing mechanical stress on the walls of the vessel and in further suppressing, preventing, or reducing the formation of bubbles.

As another example, an example catheter 102 includes an expandable mesh structure. The mesh structure encircles a portion of the catheter 104 and can be selectively expanded and collapsed. For example, after a portion of the catheter 102 has been inserted into a vessel 160, the mesh structure can be expanded such that it contacts the walls of the vessel 160. In this configuration, the catheter 104 body is positioned centrally within the vessel 150. Accordingly, the streams of gas-enriched liquid expelled by the capillaries 118a and 118b mix in a mixing region away from the walls of the vessel 160. As describe above, this can be beneficial, for example, in reducing mechanical stress on the walls of the vessel and in further suppressing preventing or reducing the formation of bubbles.

FIG. 3 is a diagram of an example catheter 300. In certain embodiments, the catheter 300 is similar to the catheter 102 of FIGS. 1A-1B. A flat end surface 140 on the distal portion 108b is polished smooth to include capillaries 118a, 118b, and 118c in a tri-capillary configuration. A channel 170 for seating a capillary 118a is shown. The catheter body 152 forms angle θ for guiding the capillaries 118a-c and thus their streams of gas-enriched liquid into the vasculature of the patient. An adhesive 150 (potting material) seats the capillaries 118a-c in place within the channels such as channel 170. In certain embodiments, the capillaries 118a-c can be slightly twisted or angled in a lateral direction so that the streams of the gas-enriched liquid swirl or twist relative to one another after being emitted from the catheter 300.

In certain embodiments, any of the capillaries 118a-c may have respective inner diameters ranging from 15-150 microns. In certain embodiments, any of the capillaries 118a-c can have respective outer diameters ranging from 50-400 microns. In certain embodiments, any of the capillaries 118a-c can have lumens with inner diameters that are sized independently from one another with the range of 15-150 microns. In certain embodiments, any of the capillaries 118a-c can have lumens with outer diameters that are sized independently from one another in the range of 50-400 microns.

For a catheter having three capillaries 118a-c configured to deliver SSO₂, when each capillary 118a-c has an inner diameter of about 50 microns, a total flow rate can be about 3 ml/min.

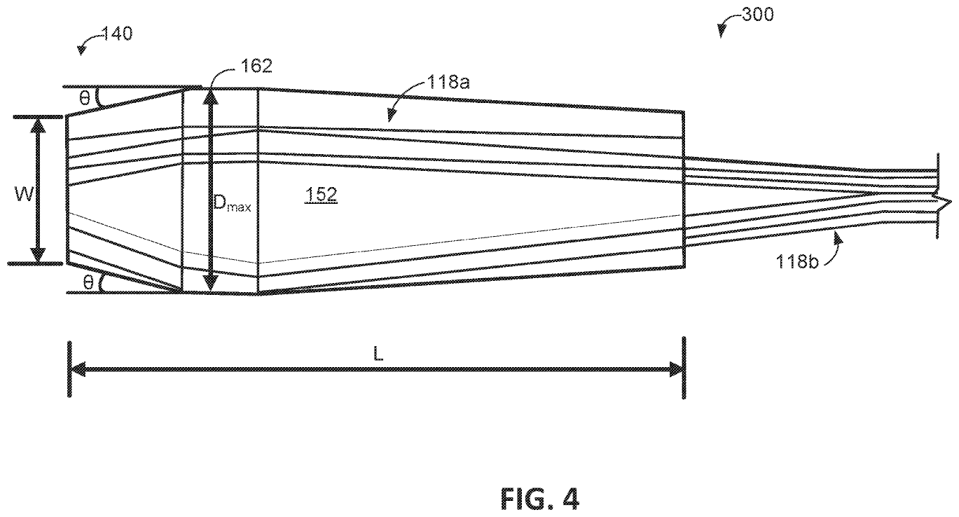

FIG. 4 is a side-view diagram of an example catheter 300, such as the catheter of FIG. 3. The catheter body 152 is shown with angles θ for the capillaries 118a-c. A length L of the catheter body 152 can be about 4 mm. A width W of the end surface 140 of the catheter 300 can be about 0.5 mm. In certain embodiments, the circumference is 3 French (Fr).

As shown in FIG. 4, the catheter 300 has a maximum diameter 162 (also labeled Dₘₐₓ), which is also called the largest diameter of the catheter. This largest diameter can be larger than a diameter of the face of the catheter 300 and can represent an upper bound on a size of the catheter, depending on into which blood vessel the catheter is inserted. The a distal end 108b maximum diameter Dₘₐₓ is about 1.15 mm or 3.5 French. In some implementations, the maximum diameter Dₘₐₓ of the catheter is between 4-7 French. In some implementations, a maximum diameter Dₘₐₓ of the catheter is between 1 to 3 French. In some implementations, a maximum diameter Dₘₐₓ of the catheter is between 1 to 7 French.

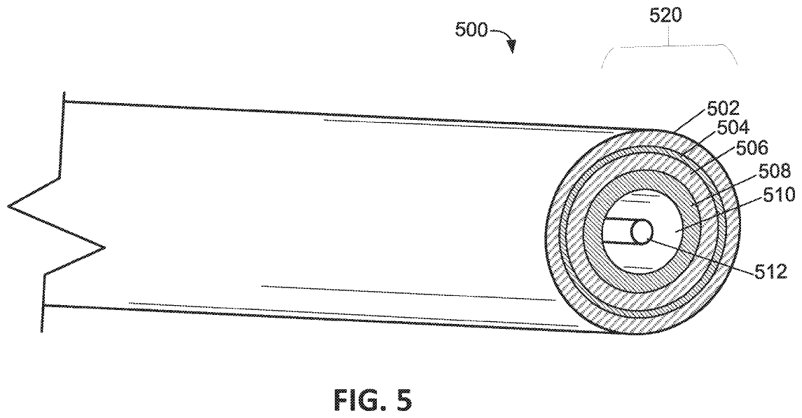

FIG. 5 is a perspective diagram of an example catheter 500. The catheter 500 can be similar to catheter 102 or catheter 300, except that catheter 500 is a single capillary catheter. For example, catheter 500 does not include a catheter body 152 or potting adhesive 150. The end 520 of the catheter 500 is polished to provide a flat or curved end surface, as previously described. The catheter 500 includes a coating 502, such as polyamide. In certain embodiments, the catheter 500 include an adhesive layer 504 to adhere to a second coating layer 506. In certain embodiments, coating layers 502 and 506 include polyamide, as previously described. Another adhesive layer 508 may surround glass walls 510. The capillary lumen 512 is at a center of the catheter 500. The catheter 500 has a small diameter (smaller than 3 Fr). The catheter 500 is flexible without breaking the glass walls 510 of the capillaries.

Example materials and diameters for catheter 500 are now described. In certain implementations, coating 502 may include a polyimide tubing that has an outer diameter of about 0.864 mm (2.6 Fr) e.g., 1 to 3 Fr, and a wall thickness of about 76 μm (0.076 mm). In this example, adhesive layer 504 includes an adhesive that runs a length of the catheter 500 and has a thickness of about 17 μm. In this example, a second coating layer 506 includes a layer of polyimide tubing and has an outer diameter of about 0.686 mm and a wall thickness of about 76 μm. In this example, the adhesive layer 508 includes a second layer of adhesive that extends about 2-5 cm from the tip of the catheter 500 and has a thickness of about 91 μm. In this example, 510 includes a silica glass capillary with an outer diameter of about 360 μm and a wall thickness of about 130 μm in diameter. In this example, a hollow center 512 (capillary) of capillary 510 is about 100 μm in diameter. For example, center 512 is the hollow center of the silica glass (capillary) and is about 100 μm in diameter. The stream of super saturated saline solution is emitted in a straight line from the tip of catheter 500. The streams of saturated solution are controlled so that the fluid flow is laminar, and there is no turbulent flow through the capillary.

In certain embodiments, the catheter 500 can be inserted in a femoral artery. In certain embodiments, the catheter 500 may be used in the venous system or in the peripheral vasculature where a low level of nucleation may be acceptable e.g., without clinically significant gas emboli). The small diameter of the catheter 500 enables these uses of the catheter. Generally, the stream of gas-enriched liquid is emitted along a catheter centerline. In certain embodiments, the catheter outer diameter is approximately 0.9 mm or 2.7 French. In certain embodiments, the catheter outer diameter is approximately 0.5 F to 1 F.

In certain embodiments, the catheter 500 has one capillary in the distal tip. The distal tip is about 125 mm long. In certain embodiments, the capillary inner diameter is about 100 μm. In certain embodiments, the capillary outer diameter is about 365 μm. In certain embodiments, the catheter 500 is about 1 meter long. Generally, the capillary is bonded, cut, and polished in place, and no glass extends beyond a tip surface. In certain embodiments, the targeted flow rate is 4.0 ml/min at 500 psi. In certain embodiments, the capillary has an inner diameter size of about 30 to 50 microns, e.g., about 40 microns. In certain embodiments, a gas-enriched liquid delivery rate can be about 0.2 to 0.7 ml/min or, e.g., about 0.5 ml/min.

In certain embodiments, the catheter 500 is configured to deliver oxygen dissolved in a liquid or solution. The catheter 500 can deliver the oxygen dissolved in the liquid at about 500-1000 pounds per square inch (PSI). In an example, the catheter 500 delivers about 0.2-3 ml O₂/ml liquid at 100-1500 PSI to prevent outgassing. In another example, the catheter 500 delivers gas-enriched liquid at 500-1000 PSI with an oxygen concentration between 1ml O₂/1ml liquid to 2ml O₂/1ml liquid. In some embodiments, the catheter 500 minimizes outgassing, as previously described. While outgassing can be limited to 0 milliliters, a maximum amount of outgassing can be no more than 0.99 ml of oxygen out of the liquid, when the gas-enriched liquid is delivered into tissue. In another embodiment, the catheter 500 limits outgassing to no more than 0.01 milliliters of oxygen out of the liquid.

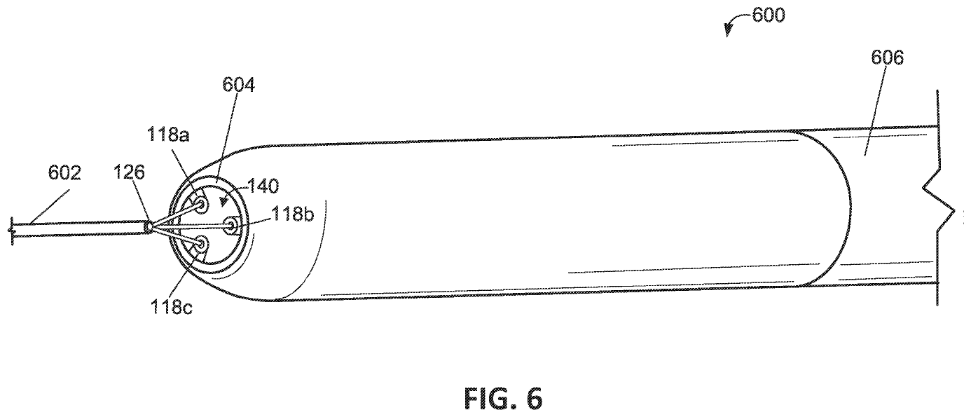

FIG. 6 is a diagram of an example catheter 600. In certain embodiments, the catheter 600 is similar to catheter 102 of FIG. 1A. The catheter 600 has three capillaries 118a-c. The catheter 600 includes an overmold material 604 that encapsulates the catheter body 152 and the capillaries 118a-c in their channels 170. The overmold 604 is a layer of material that is soft (such as rubber or a plastic). The overmold 604 is configured to soften the catheter 600 and reduce or eliminate a risk for damage to a vasculature of the patient. A body 606 of the catheter 600 extends beyond the overmold 604 material.

In certain embodiments, the angle θ of the three jets from a center line of the catheter 600 is about 16.5°. In certain embodiments, the over-molded tip diameter is about 1.3 mm or 4.0 French. In certain embodiments, the three capillaries 118a-c are about 24 mm long. The capillaries 118a of the catheter have an inner diameter of about 50 μm. In certain embodiments, the capillaries 118a-c have an outer diameter of about 150 μm. In certain embodiments, the catheter 600 is about 1 meter long. In certain embodiments, the catheter 600 has an outer diameter of about 1.3 mm or 4.0 French. In certain embodiments, the distal end 108b (tip) end surface 140 face is bonded with epoxy and polished, and no glass extends beyond a tip end surface 140 or 142.

Streams of the gas-enriched liquid are shown as emitted from the respective capillaries 118a-c to intersect at mixing region 126. A stream 602 of combined gas-enriched liquid is formed.

As described above, the capillaries of a catheter can be configured such that the streams of gas-enriched liquid expelled by the capillaries mix in a mixing region within the vasculature of the patient. For example, the capillaries can define respective paths that are angled relative to the longitudinal axis of the catheter, such that the streams are expelled from the capillaries at respective angles relative to the longitudinal axis 106. In certain embodiments, this angle can be from 10 degrees to 80 degrees or from 15 degrees to 75 degrees. The catheter may be inserted in one or more coronary arteries, peripheral vasculature or other vessel.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable sub-combination.

A number of embodiments have been described. Nevertheless, various modifications can be made without departing from the spirit and scope of the invention. Accordingly, other embodiments are within the scope of the claims.

What is claimed is:

1. A catheter configured to be inserted into a vasculature of a patient for delivery of a gas-enriched liquid, the catheter comprising:
a catheter body; and
two or more capillaries coupled to the catheter body and configured to receive a gas-enriched liquid from a gas-enriched liquid source;
wherein an end surface of the catheter includes a first end of the catheter body and first ends of each of the two or more capillaries, the first ends of the two or more capillaries having apertures through which the gas-enriched liquid can flow, the first ends of the two or more capillaries being flush with the first end of the catheter body wherein the end surface of the catheter is smooth and includes the apertures of the two or more capillaries;
the two or more capillaries being in fluid communication with the vasculature of the patient when the catheter body is inserted into the vasculature of the patient;
the two or more capillaries being configured to dispense respective streams of the gas-enriched liquid directly into the vasculature of the patient; and
the first ends of the two or more capillaries positioned relative to one another to cause the streams of the gas-enriched liquid to intersect and mix with blood of the patient in a region beyond the first end of the catheter body.

2. The catheter of claim 1, wherein the end surface is a flat surface.

3. The catheter of claim 1, wherein the end surface is a concave surface.

4. The catheter of claim 1, wherein the first ends of the two or more capillaries are beveled.

5. The catheter of claim 1, wherein the catheter body includes slots for positioning the two or more capillaries, the slots positioning the two or more capillaries with respect to one another for causing the streams of the gas-enriched liquid to intersect.

6. The catheter of claim 1, the two or more capillaries being coupled to the catheter body by an adhesive.

7. The catheter of claim 1, wherein a capillary of the two or more capillaries comprises a glass wall that is coated in a polyamide.

8. The catheter of claim 7, wherein the glass wall is polished to form a first end that is a portion of the end surface.

9. The catheter of claim 1, wherein the gas-enriched liquid comprises a supersaturated oxygen enriched liquid.

10. The catheter of claim 1, further comprising a lumen configured to receive a guide wire for positioning the catheter with respect to the vasculature of the patient.

11. The catheter of claim 10, further comprising a pressure sensor disposed within the lumen, wherein the pressure sensor is configured to obtain one or more pressure measurements.

12. The catheter of claim 1, wherein a diameter of the end surface is between 1 and 14 French.

13. The catheter of claim 1, wherein the catheter comprises:
a lumen extending through the catheter body, wherein the lumen is configured to be in fluid communication with the vasculature of the patient when the catheter is inserted into the vasculature of the patient, and the lumen is configured to perform at least one of receiving an additional sample of the blood from the vasculature of the patient and or measuring an additional parameter of the blood.

14. The catheter of claim 1, wherein the catheter body is configured to position the two or more capillaries such that the streams of the gas-enriched liquid intersect a longitudinal axis extending through a center of the catheter.

15. The catheter of claim 1, wherein the catheter body is configured to position the two or more capillaries such that the streams of the gas-enriched liquid do not intersect a longitudinal axis extending through a center of the catheter.

16. The catheter of claim 1, wherein the two or more capillaries form respective lumens that are angled with respect a longitudinal axis of the catheter body at an angle of between 10-30 degrees.

17. The catheter of claim 1, wherein a diameter of at least one of the two or more capillaries and a diameter of at least another one of the two or more capillaries are equal.

18. The catheter of claim 1, wherein a diameter of at least one of the two or more capillaries is different from a diameter of at least another one of the two or more capillaries.

19. The catheter of claim 1, wherein at least one of the two or more capillaries has an inner diameter that ranges from 25 microns to 400 microns.

20. The catheter of claim 1, wherein at least one of the two or more capillaries has an outer diameter that ranges from 30 microns to 1000 microns.

21. The catheter of claim 1, wherein a distance between (i) an intersection of the streams of the gas-enriched liquid and (ii) an end tip of the catheter or an output aperture of a through lumen is greater than or equal to a diameter of the end tip of the catheter.

22. The catheter of claim 1, wherein a distance between (i) the first ends of the two or more capillaries and (ii) an end tip of the catheter or an output aperture of a first lumen is greater than or equal to a diameter of the end tip of the catheter.

23. The catheter of claim 1, wherein at least one capillary comprises a fiducial marking.

24. The catheter of claim 1, wherein the catheter body is configured to position the two or more capillaries such that the streams of the gas-enriched liquid intersect and mix with the blood without formation of bubbles.

25. The catheter of claim 1, wherein the catheter body is configured to position the two or more capillaries such that the streams of the gas-enriched liquid minimize outgassing to less than a maximum of 1 milliliter of outgassing of dissolved oxygen from the gas-enriched liquid.

26. The catheter of claim 1, wherein the catheter body is configured to position the two or more capillaries such that the streams of the gas-enriched liquid minimize outgassing to a maximum of 0.01 milliliters of outgassing of dissolved oxygen from the gas-enriched liquid.

27. The catheter of claim 1, wherein the streams of the gas-enriched liquid intersect and mix with the blood in a region beyond an end of the catheter body to reduce bubble nucleation along one or more surfaces of the catheter body or at least two capillaries.

28. The catheter of claim 1, further comprising one or more sensors, wherein the one or more sensors are configured such that the one or more sensors are in fluid communication with the vasculature of the patient when the catheter is inserted into the vasculature of the patient.

29. The catheter of claim 28, wherein at least one of the one or more sensors is an oxygen partial pressure (pO₂) sensor.

30. The catheter of claim 1, further comprising a self-centering device configured to center the catheter body within the vasculature of the patient.

31. The catheter of claim 30, wherein the self-centering device comprises one or more mesh structures encircling the catheter body.

32. The catheter of claim 30, wherein the self-centering device comprises a balloon.

33. The catheter of claim 1, further comprising a cover that encloses the catheter body and the two or more capillaries, the cover comprising a soft rubber.

34. The catheter of claim 1, wherein the catheter body comprises a polycarbonate material.

35. The catheter of claim 1, wherein the two or more capillaries comprise three capillaries, the catheter further comprising:
the catheter body comprising, for each capillary of the three capillaries, a channel in which that capillary is seated, the channel comprising a straight portion and an angled portion, the angled portion causing that capillary to form an angle with respect to a longitudinal axis of the catheter body, the catheter body comprising a flat surface at a distal portion of the catheter body;
wherein each capillary is seated in a potting material, the potting material holding that capillary in place with respect to the catheter body,
wherein each capillary comprises a glass wall coated in a polyamide coating,
wherein, for each capillary, the polyamide coating and the glass wall are polished to a flat surface that is flush with the flat surface of the catheter body,
wherein the capillaries are each angled at about 10-30° with respect to the longitudinal axis, and
wherein streams of the gas-enriched liquid emitted from each of the capillaries are configured to mix at about 0.5 mm from the flat surface of the catheter body.

36. A method for forming a catheter for delivering a gas-enriched liquid, the method comprising:
forming a catheter body configured for placement in a vasculature of a patient, the catheter body comprising two or more channels each configured for receiving capillaries that deliver a gas-enriched liquid to the vasculature of the patient when the catheter body is placed in the vasculature of the patient;
coupling two or more capillaries to the catheter body, the two or more capillaries being configured to receive a gas-enriched liquid from a gas-enriched liquid source; and
forming an end surface including a first end of the catheter body and first ends of each of the two or more capillaries, the first ends of the two or more capillaries having apertures through which the gas-enriched liquid can flow, the first ends of the two or more capillaries being flush with the first end of the catheter body so that the end surface of the catheter is smooth and includes the apertures for the two or more capillaries,
the first ends of the two or more capillaries positioned relative to one another to cause streams of the gas-enriched liquid to intersect and mix with blood of the patient in a region beyond the first end of the catheter body.

37. The method of claim 36, wherein forming an end surface including a first end of the catheter body and first ends of each of the two or more capillaries comprises:
removing a polyamide coating from the first sends of the two or more capillaries; and
polishing the catheter body and the two or more capillaries together to form the end surface that is smooth.

38. The method of claim 37, wherein the polishing is performed using a flat surface.

39. The method of claim 37, wherein the polishing is performed using a curved surface.

40. The method of claim 36, wherein the two or more capillaries are configured such that the streams of the gas-enriched liquid minimize outgassing to less than a maximum of 1 milliliter of outgassing of dissolved oxygen from the gas-enriched liquid.

41. The method of claim 36, wherein the two or more capillaries are configured such that the streams of the gas-enriched liquid minimize outgassing to a maximum of 0.01 milliliters of outgassing of dissolved oxygen from the gas-enriched liquid.

42. A catheter for delivering a gas-enriched liquid comprising:
a single capillary comprising a lumen formed by a glass wall, the single capillary including a first end, wherein the first end of the single capillary has an aperture through which a stream of gas-enriched liquid can flow;
the single capillary being coated in a first coating layer, the first coating layer being adhered to a second coating layer by an adhesive, and
wherein the first coating layer, the second coating layer, the glass wall, and the adhesive are polished to form a flat, continuous surface comprising the aperture.

43. The catheter of claim 42, wherein the capillary is configured such that the stream of the gas-enriched liquid minimizes outgassing to less than a maximum of 1 milliliter of outgassing of dissolved oxygen from the gas-enriched liquid into a vasculature of a patient.

44. The catheter of claim 42, wherein the capillary is configured such that the stream of the gas-enriched liquid minimizes outgassing to a maximum of 0.01 milliliters of outgassing of dissolved oxygen from the gas-enriched liquid into a vasculature of a patient.

45. The catheter of claim 42, wherein an outer diameter of a catheter body that supports the single capillary is 0.5 to 3 French.

46. The catheter of claim 42, wherein an outer diameter of a catheter body that supports the single capillary is between 0.5 to 1 French.

47. The catheter of claim 42, wherein the capillary has an inner diameter size of between 30 and 50 microns.

48. The catheter of claim 42, wherein an SSO₂ delivery rate from the capillary is configured to be between 0.25-0.75 ml/min.

49. The catheter of claim 42, wherein the capillary is configured such that the stream of the gas-enriched liquid minimizes outgassing to less than a maximum of 1 milliliters outgassing of dissolved oxygen from the gas-enriched liquid into a vasculature of a patient.

50. The catheter of claim 42, wherein the capillary is configured such that the stream of the gas-enriched liquid minimizes outgassing to a maximum of 0.01 milliliters of outgassing of dissolved oxygen from the gas-enriched liquid into a vasculature of a patient.

51. The catheter of claim 1, comprising a conical end surface of the catheter and wherein the first ends or apertures of the two or more capillaries are positioned such that the streams from the two or more capillaries are substantially perpendicular relative to the first end of the catheter body.

52. The catheter of claim 1, comprising a curved, spherical end surface of the catheter and wherein the first ends or apertures of the two or more capillaries are positioned such that the streams from the two or more capillaries are substantially perpendicular relative to the first end of the catheter body.

53. The catheter of claim 1, wherein a diameter of the end surface is between 1 and 6 French.

54. The catheter of claim 1, wherein a diameter of the end surface is between 1 and 3 French.

55. The catheter of claim 1, wherein an inner diameter of at least one of the two or more capillaries is between 15-150 microns.

56. The catheter of claim 1, wherein an outer diameter of at least one of the two or more capillaries is between 50-400 microns.

57. The catheter of claim 1, wherein an inner diameter of at least one of the two or more capillaries is between 40-60 microns.

58. The catheter of claim 1, wherein a gas-enriched liquid delivery rate from the two or more capillaries is between 2-4 ml/min.

59. The catheter of claim 1, wherein a gas-enriched liquid delivery rate from the two or more capillaries is between 0.1-20 ml/min.

60. The catheter of claim 1, wherein the catheter body is configured to position the two or more capillaries such that the streams of the gas-enriched liquid minimizes outgassing to less than a maximum of 1 milliliters of gas or outgassing of dissolved oxygen from the gas-enriched liquid.

61. The catheter of claim 1, wherein the catheter body is configured to position the two or more capillaries such that the streams of the gas-enriched liquid minimizes outgassing to a maximum of 0.01 milliliters of outgassing of dissolved oxygen from the gas-enriched liquid.

62. The catheter of claim 1, wherein the two or more capillaries are configured to deliver the streams of the gas-enriched liquid to deliver oxygen dissolved in liquid at an oxygen concentration of 1 ml/ml to 2 ml/ml.

63. The catheter of claim 62, wherein the streams of the gas-enriched liquid are configured to deliver the oxygen dissolved in liquid with a maximum outgassing of 0.01 milliliters of gas.

64. The catheter of claim 62, wherein the streams of the gas-enriched liquid are configured to deliver the oxygen dissolved in liquid with a maximum outgassing of less than 1 milliliter of gas.

65. The catheter of claim 1, wherein the two or more capillaries form respective lumens that are angled with respect to a longitudinal axis of the catheter body at an angle between 10-30 degrees.

66. The catheter of claim 1, wherein each of the two or more capillaries are in fluid communication with a respective lumen, and wherein each of the two or more capillaries lumen is sized to enable gas-enriched liquid delivery rates between 0.1-20 ml/min.

67. The catheter of claim 1, wherein a minimum flow rate through each of the two or more capillaries ensures the gas-enriched liquid sprays out of a respective tip for each of the two or more capillaries.

68. The catheter of claim 1, wherein the minimum flow rate enables a Reynolds number of about 1000.

69. The catheter of claim 1, wherein each of the two or more capillaries comprises an inner diameter size of between 25-35 microns and is configured for a delivery rate of 0.25-1.0 ml/min at 37° C. and about 500 pounds per square inch of pressure.

70. The catheter of claim 1, wherein each of the two or more capillaries comprises an inner diameter size of between 45-55 microns and is configured for a delivery rate of 0.4-1.6 ml/min at 37° C. and about 500 pounds per square inch of pressure.

71. The catheter of claim 42, the single capillary comprises an inner diameter size of between 25-35 microns and is configured for a delivery rate of 0.25-1.0 ml/min at 37° C. and about 500 pounds per square inch of pressure.

72. The catheter of claim 42, wherein the single capillary comprises an inner diameter size of between 45-55 microns and is configured for a delivery rate of 0.4-1.6 ml/min at 37° C. and about 500 pounds per square inch of pressure.

73. The catheter of claim 1, wherein the two or more capillaries comprise three capillaries, the three capillaries each having an inner diameter of about 25-35 microns for a total flow rate can be about 0.5 to 3 ml/min.

74. The catheter of claim 1, wherein the two or more capillaries comprise three capillaries, the three capillaries each having an inner diameter of about 45-55 microns for a total flow rate can be about 1 to 5 ml/min.

75. A system comprising:
the catheter of any of claims 1 to 35;
a gas-enriched liquid source; and
one or more pumps configured to provide the gas enriched liquid from the gas-enriched liquid source to the two or more capillaries.

76. A system comprising:
the catheter of any of claims 42 to 74;
a gas-enriched liquid source; and
one or more pumps configured to provide the gas enriched liquid from the gas-enriched liquid source to the capillary.
